(19) United States
(12) Patent Application Publication
(10) Pub. No.: US 2022/0025058 A1
(43) Pub. Date: Jan. 27, 2022

(54) METHODS AND PHARMACEUTICAL COMPOSITIONS FOR THE TREATMENT OF ACUTE MYELOID LEUKEMIA BY ERADICATING LEUKEMIC STEM CELLS

(71) Applicants: INSERM (INSTITUT NATIONAL DE LA SANTÉ ET DE LA RECHERCHE MÉDICALE), Paris (FR); UNIVERSITÉ PAUL SABATIER TOULOUSE III, Toulouse (FR); CENTRE HOSPITALIER UNIVERSITAIRE DE TOULOUSE

(72) Inventors: Jean-Emmanuel SARRY, Toulouse (FR); Clément LARRUE, Toulouse (FR); Christian RECHER, Toulosue (FR)

(21) Appl. No.: 17/291,796

(22) PCT Filed: Nov. 5, 2019
(86) PCT No.: PCT/EP2019/080174

§ 371 Date: May 6, 2021

(30) Foreign Application Priority Data
Nov. 6, 2018 (EP) 18306452.6
Nov. 22, 2018 (EP) 18306549.9

(51) Int. Cl. C07K 16/28; A61P 35/02; A61K 47/68; G01N 33/574

(52) U.S. Cl. CPC C07K 16/2869; A61P 35/02; A61K 47/6849; C07K 2317/31; C07K 2317/732; C07K 2317/24; C07K 2317/734; G01N 33/57426

(57) ABSTRACT

After intensive chemotherapy, the emergence of cells with dmg resistant and/or stem cell features might explain frequent relapses and the poor outcome of patients with acute myeloid leukemia (AML). Herein the inventors first uncovered that the adrenomedullin receptor CALCRL is overexpressed in AML patients comparing with normal cells and preferentially in the immature CD34⁺ CD38⁻ compartment. Then they demonstrated its role in the maintenance of leukemic stem cell function in vivo. Moreover, CALCRL depletion strongly affected leukemic growth in xenograft models and sensitized to chemotherapeutic agent cytarabine in vivo. It Accordingly, the inventors showed that ADM-CALCRL axis drove cell cycle, DNA integrity, and high OxPHOS status of chemoresistant AML stem cells in an E2F1- and BCL2-dependent manner. Furthermore, CALCRL depletion sensitizes cells to cytarabine and its CT expression predicted the response to chemotherapy in vivo in mice. Further, using the combination of limiting dilution assays, single-cell RNA-seq analysis of primary AMF samples at diagnosis and relapse and before and after transplantation in NSG mice, the inventors revealed the pre-existence of a chemoresistant leukemic stem cell sub-population harboring a CALCRL-driven gene signature. Finally the inventors strongly demonstrated that chemoresistant LSC are dependent for CALCRL. All of these data highlight the critical role of CALCRL in stem cell survival, proliferation and metabolism and identify this receptor as a new marker of chemoresistant leukemic stem cell population and a promising therapeutic target to specifically eradicate them and overcome relapse in AML.

### FIELD OF THE INVENTION:

The present invention relates to methods and pharmaceutical compositions for the treatment of acute myeloid leukemia (AML) by eradicating leukemic stem cells.

### BACKGROUND OF THE INVENTION:

Acute myeloid leukemia (AML) arises from self-renewing leukemic stem cells (LSCs) which can repopulate human AML when assayed and xenografted in immunocompromised mice (e.g. PDX; Bonnet and Dick, 1997). Despite the fact that these cells represent a tiny minority of all leukemic cells, the fact that gene signatures associated with a stem cell phenotype or function are associated with an unfavorable prognosis in AML, strongly supports the hypothesis that their abundance has a real clinical impact (Gentles et al., 2010; Vergez et al., 2011; Eppert et al., 2011; Ng et al., 2016). This clinical relevance is supported by studies showing that relapsing patients presents a constantly enrichment of LSC frequency (Ho et al., 2016) and an increase of LSC-related gene signatures (Hackl et al., 2015). While it has been initially shown that LSCs are spared from chemotherapy (Jordan et al., 2006; Ishikawa et al., 2007), recent works demonstrated that cytarabine (AraC) can have a strong impact on the LSC pool in PDX models and patients (Farge et al., 2017; Boyd et al., 2018). These results suggest that there are two distinct LSC populations, some chemosensitive (S-LSCs) and thus eradicated by conventional treatments, and others that are chemoresistant (R-LSCs) persist, regenerate AML and initiate the relapse of patients. Thus, better phenotypically and functionally characterizing these R-LSCs is crucial to allow the development of new therapy strategies that specifically target R-LSCs.

Although it has been first proposed that the LSC-compartment was restricted to the CD34⁺ CD38⁻ subpopulation of human AML cells (Bonnet and Dick, 1997; Ishikawa et al., 2007), several studies subsequently demonstrated that LSCs are also phenotypically heterogeneous such as for instance CD38⁺ AML cells or CD34⁻ cells from NPM1c-mutated specimens are also able to serially recapitulate the disease when assayed in NSG-deficient mice (Taussig et al., 2008; Taussig et al., 2010; Sarry et al., 2011; Quek et al., 2016). This highlights that more functional studies are needed to well characterize LSCs (Eppert et al., 2011). Eradicating R-LSCs without killing normal hematopoietic stem cells (HSCs) depends on identifying markers overexpressed in AML compartment and functionally relevant. In recent years, many research efforts to distinguish LSCs from HSCs have been made and allowed the identification of several cell surface markers such as CD47, CD123, CD44, TIM-3, CD25, CD32 or CD93 (Majeti et al., 2009; Jin et al., 2009; Kikushige et al., 2010; Saito et al., 2010; Iwasaki et al., 2015). In addition to these new membrane markers, it has been proposed that LSCs have also a specific increase in BCL2-dependent oxidative phosphorylation (OxPHOS), revealing a Achille' s heel (vulnerability) that could be exploited through the treatment with BCL2 inhibitors such as ABT-199 (Lagadinou et al., 2013; Konopleva et al., 2016). This is consistent with several studies including our recent one that demonstrate that mitochondrial OxPHOS status contributes to drug resistance in leukemia (Farge et al., 2017; Bosc et al. 2017; Kunst et al. 2017). Taken together, all these results suggest specific characteristics of R-LSCs that leave an open door to targeted therapeutic approaches aimed at eradicating these cells.

### SUMMARY OF THE INVENTION:

The present invention relates to methods and pharmaceutical compositions for the treatment of acute myeloid leukemia (AML) by eradicating leukemic stem cells. In particular, the present invention is defined by the claims.

### DETAILED DESCRIPTION OF THE INVENTION:

After intensive chemotherapy, the emergence and the persistence of AML cells with drug resistant and/or stem cell features might explain frequent relapses and the poor outcome of patients with acute myeloid leukemia (AML). Herein the inventors first uncovered that the adrenomedullin receptor CALCRL is overexpressed in AML patients comparing with normal cells and preferentially in the immature CD34⁺ CD38⁻ compartment. Then they demonstrated its role in the maintenance of leukemic stem cell function in vivo. Moreover, CALCRL depletion strongly affected leukemic growth in xenograft models and sensitized to chemotherapeutic agent cytarabine in vivo. Accordingly, the inventors showed that ADM-CALCRL axis drove cell cycle, DNA integrity, and high OxPHOS status of chemoresistant

AML stem cells in both an E2F1- and BCL2-dependent manner. Furthermore, CALCRL depletion sensitizes cells to cytarabine and its expression predicted the response to chemotherapy in vivo in mice. Further, using the combination of limiting dilution assays, single-cell RNA-seq analysis of primary AML samples at diagnosis and relapse and before and after transplantation in NSG mice, the inventors revealed the pre-existence of a chemoresistant leukemic stem cell sub-population harboring a CALCRL-driven gene signature. Finally the inventors strongly demonstrated that chemoresistant LSC are dependent for CALCRL. All of these data highlight the critical role of CALCRL in stem cell survival, proliferation and metabolism and identify this receptor as a new biomarker of chemoresistant leukemic stem cell population and a promising therapeutic target to specifically eradicate them and overcome relapse in AML.

Accordingly, the first object of the present invention relates to a method of depleting leukemic stem cells in a subject suffering from AML comprising administering to the subject a therapeutically effective amount of an antibody that specifically binds to CALCRL thereby depleting said leukemic stem cells.

A further object of the present invention relates to a method of depleting leukemic stem cells in a subject suffering from AML comprising administering to the subject a therapeutically effective amount of an inhibitor of CALCRL activity or expression thereby depleting said leukemic stem cells.

As used herein, the term “acute myeloid leukemia” or “acute myelogenous leukemia” (“AML”) refers to a cancer of the myeloid line of blood cells, characterized by the rapid growth of abnormal white blood cells that accumulate in the bone marrow and interfere with the production of normal blood cells.

As used herein, the term “leukemic stem cell” has its general meaning in the art and refers to a pluripotent myeloid stem cell characterized by genetic transformation resulting in unregulated cell division. The leukaemic stem cells (LSC) are distinguished from all other AML cells by self-renewal ability, i.e. the ability to generate daughter cells similar to the mother one. The extensive self-renewal ability is an intrinsic property of LSC, and has been shown essential for the development of leukaemia.

The method of the present invention is thus particularly suitable for the treatment of AML.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

The method of the present invention is particularly suitable for preventing relapse of a patient suffering from AML who was treated with chemotherapy.

As used herein, the term “relapse” refers to the return of cancer after a period of improvement in which no cancer could be detected. Thus, the method of the present invention is particularly useful to prevent relapse after putatively successful treatment with chemotherapy.

Accordingly, a further object of the present invention relates to a method of treating chemoresistant acute myeloid leukemia (AML) in a patient in need thereof comprising administering to the patient a therapeutically effective amount an antibody that specifically binds to CALCRL.

Accordingly, a further object of the present invention relates to a method of treating chemoresistant acute myeloid leukemia (AML) in a patient in need thereof comprising administering to the patient a therapeutically effective amount of an inhibitor of CALCRL activity or expression.

As used herein; the term “chemoresistant acute myeloid leukemia” refers to the clinical situation in a patient suffering from acute myeloid leukemia when the proliferation of cancer cells cannot be prevented or inhibited by means of a chemotherapeutic agent or a combination of chemotherapeutic agents usually used to treat AML, at an acceptable dose to the patient. The leukemia can be intrinsically resistant prior to chemotherapy, or resistance may be acquired during treatment of leukemia that is initially sensitive to chemotherapy.

As used herein, the term “chemotherapeutic agent” refers to any chemical agent with therapeutic usefulness in the treatment of cancer. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these drugs are directly toxic to cancer cells and do not require immune stimulation. Suitable chemotherapeutic agents are described, for example, in Slapak and Kufe, Principles of Cancer Therapy, Chapter 86 in Harrison's Principles of Internal medicine, 14th edition; Perry et at , Chemotherapeutic, Ch 17 in Abeloff, Clinical Oncology 2nd ed., 2000 ChrchillLivingstone, Inc.; Baltzer L. and Berkery R. (eds): Oncology Pocket Guide to Chemotherapeutic, 2nd ed. St. Louis, mosby-Year Book, 1995; Fischer D. S., Knobf M. F., Durivage H J. (eds): The Cancer Chemotherapeutic Handbook, 4th ed. St. Louis, Mosby-Year Handbook.

In some embodiments the chemotherapeutic agent is cytarabine (cytosine arabinoside, Ara-C, Cytosar-U), quizartinib (AC220), sorafenib (BAY 43-9006), lestaurtinib (CEP-701), midostaurin (PKC412), carboplatin, carmustine, chlorambucil, dacarbazine, ifosfamide, lomustine, mechlorethamine, procarbazine, pentostatin, (2′deoxycoformycin), etoposide, teniposide, topotecan, vinblastine, vincristine, paclitaxel, dexamethasone, methylprednisolone, prednisone, all- trans retinoic acid, arsenic trioxide, interferon-alpha, rituximab (Rituxan®), gemtuzumab ozogamicin, imatinib mesylate, Cytosar-U), melphalan, busulfan (Myleran®), thiotepa, bleomycin, platinum (cisplatin), cyclophosphamide, Cytoxan®)., daunorubicin, doxorubicin, idarubicin, mitoxantrone, 5-azacytidine, cladribine, fludarabine, hydroxyurea, 6-mercaptopurine, methotrexate, 6-thioguanine, or any combination thereof. In some embodiments, the leukemia is resistant to a combination of daunorubicin, or idarubicin plus cytarabine (AraC).

In some embodiments, the chemotherapeutic agent is a BCL2 inhibitor. In some embodiments, the Bcl-2 inhibitor comprises 4-(4-{ [2-(4-chlorophenyl)-4,4-dimethylcyclohex-1-en-1- yl] methyl }piperazin-1-yl)-N-({3-nitro-4- Rtetrahydro-2H-pyran-4-ylmethyl)aminolphenyl}sulfonyl)-2- (1H-p yrrolo [2,3-b]pyridin-5-yloxy)benzamide (also known as, and optionally referred to herein as, venetoclax, or ABT-199, or GDC-0199) or a pharmaceutically acceptable salt thereof.

In some embodiments, the chemotherapeutic agent is a FLT3 inhibitor. Examples of FLT3 inhibitors include N-(2- diethylaminoethyl)-5 -[(Z)-(5-fluoro-2-oxo- 1 H-indol-3 -ylidene)methyl]-2,4-dimethyl-1H-pyrrole-3-carboxamide, sunitinib, also know as SU11248, and marketed as SUTENT (sunitinib malate) ; 4-[4-[ [4-chloro-3 -(trifluoromethyl)phenyl] carbamoylamino]phenoxy] -N-methyl-pyridine-2-carboxamide, sorafenib, also known as BAY 43-9006, marketed as NEXAVAR (sorafenib); (9S,10R,1 1R,13R)-2,3, 10,11, 12,13-Hexahydro-10-methoxy-9-methyl-1 1-(methylamino)-9,13-epoxy-1H,9H-diindolo [1,2,3-gh:3′,2′,1′-1m]pyrrolo[3,4- j][1,7]benzodiamzonine-l-one, also know as midostaurin or PKC412; (5S,6S,8R)-6-Hydroxy-6- (hydroxymethyl)-5-methyl-7,8,14,15-tetrahydro-5H-16-oxa-4b,8a,14-triaza-5,8- methanodibenzo [b,h]cycloocta[jkl]cyclopenta[e]-as-indacen-13(6H)-one, also know as lestaurtinib or CEP-701; 1-(5-(tert-Butyl)isoxazol-3-yl)-3-(4-(7-(2-morpholinoethoxy)benzo[d]imidazo[2,1-b]thiazol-2-yl)phenyl)urea, also known as Quizartinib or AC220; 1- (2-{5-[(3-Methyloxetan-3-yl)methoxy]-1H-benzimidazol-1-yl}quinolin-8-yl)piperidin-4-amine, also known as Crenolanib or CP-868,596-26. See, e.g., Wander S. A., TherAdv Hematol. 5: 65-77 (2014). Other FLT3 inhibitors include Pexidartinib (PLX-3397), Tap et al, N Engl J Med, 373:428-437 (2015); gilteritinib (ASP2215), Smith et al., Blood: 126 (23) (2015); FLX-925, also known as AMG-925, Li et al. Mol. Cancer Ther. 14: 375-83 (2015); and G-749, Lee et al, Blood. 123: 2209-2219 (2014).

In some embodiments, the chemotherapeutic agent is an IDH (isocitrate dehydrogenase) inhibitor. In some embodiments, the IDH inhibitor is a member of the oxazolidinone (3-pyrimidinyl-4-yl- oxazolidin-2-one) family, and is a specific inhibitor of the neomorphic activity of IDH1 mutants and has the chemical name (S)-4-isopropyl-3-(2- (((S)-1-(4 phenoxyphenyl)ethyl)amino)pyrimidin-4-yl)oxazolidin-2-one.

As used herein the term “CALCRL” has its general meaning in the art and refers to calcitonin receptor like receptor (Gene ID: 10203). CALCRL is also named CRLR or CGRPR. CALCRL is linked to one of three single transmembrane domain receptor activity-modifying proteins (RAMPs) that are essential for functional activity. The association of CALCRL with different RAMP proteins produces different receptors: i) with RAMPl: produces a CGRP receptor, ii) with RAMP2: produces an adrenomedullin (AM) receptor, designated AM1, and iii) with RAMP3: produces a dual CGRP/AM receptor designated AM2. These receptors are linked to the G protein Gs which activates adenylate cyclase and activation results in the generation of intracellular cyclic adenosine monophosphate (cAMP). An exemplary amino acid sequence of CALCRL is represented by SEQ ID NO: 1.

>sp|Q16602|CALRL_HUMAN Calcitonin
gene-related peptide type 1 receptor
OS = Homo sapiens OX = 9606
GN = CALCRL PE = 1 SV = 2
SEQ ID NO: 1
MEKKCTLNFLVLLPFFMILVTAELEESPED
SIQLGVTRNKIMTAQYECYQKIMQDPIQQA
EGVYCNRTWDGWLCWNDVAAGTESMQLCPD
YFQDFDPSEKVTKICDQDGNWFRHPASNRT
WTNYTQCNVNTHEKVKTALNLFYLTIIGHG
LSIASLLISLGIFFYFKSLSCQRITLHKNL
FFSFVCNSVVTIIHLTAVANNQALVATNPV
SCKVSQFIHLYLMGCNYFWMLCEGIYLHTL
IVVAVFAEKQHLMWYYFLGWGFPLIPACIH
AIARSLYYNDNCWISSDTHLLYIIHGPICA
ALLVNLFFLLNIVRVLITKLKVTHQAESNL
YMKAVRATLILVPLLGIEFVLIPWRPEGKI
AEEVYDYIMHILMHFQGLLVSTIFCFFNGE
VQAILRRNWNQYKIQFGNSFSNSEALRSAS
YTVSTISDGPGYSHDCPSEHLNGKSIHDIE
NVLLKPENLYN

As used herein, the term “deplete” with respect to leukemic stem cells, refers to a measurable decrease in the number of leukemic stem cells in the subject. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of leukemic stem cells in a subject or in a sample to an amount below detectable limits.

According to the present invention, the antibody specifically mediates depletion of the leukemic stem cell subset populations and do not mediate depletion of population of hematopoietic cells.

As used herein, the term “antibody” has its general meaning in the art and encompasses monoclonal antibodies (including full-length monoclonal antibodies), polyclonal antibodies, multispecific antibodies (e.g., bispecific antibodies) formed from at least two intact antibodies, human antibodies, humanized antibodies, camelised antibodies, chimeric antibodies, single-chain Fvs (scFv), single-chain antibodies, single domain antibodies, domain antibodies, Fab fragments, F(ab')2 fragments, antibody fragments that exhibit the desired biological activity, disulfide-linked Fvs (sdFv), and anti-idiotypic (anti-Id) antibodies (including, e.g., anti-Id antibodies to antibodies of the invention), intrabodies, and epitope-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

According to the present invention, the antibody binds to at least one extracellular domain of CALCRL.

As used herein the term “bind” indicates that the antibody has affinity for the surface molecule. The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

In some embodiments, the antibody of the present invention is a monoclonal antibody. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, monoclonal antibodies are advantageous in that they can be synthesized by hybridoma cells that are uncontaminated by other immunoglobulin producing cells. Alternative production methods are known to those trained in the art, for example, a monoclonal antibody may be produced by cells stably or transiently transfected with the heavy and light chain genes encoding the monoclonal antibody.

Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with the appropriate antigenic forms (i.e. polypeptides of the present invention). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Briefly, the recombinant polypeptide of the present invention may be provided by expression with recombinant cell lines. Recombinant forms of the polypeptides may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods. Following culture of the hybridomas, cell supernatants are analysed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

In some embodiments, the monoclonal antibody of the invention is a chimeric antibody, in particular a chimeric mouse/human antibody. As used herein, the term “chimeric antibody” refers to an antibody which comprises a VH domain and a VL domain of a non-human antibody, and a CH domain and a CL domain of a human antibody. In some embodiments, the human chimeric antibody of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. As the CH domain of a human chimeric antibody, it may be any region which belongs to human immunoglobulin, but those of IgG class are suitable and any one of subclasses belonging to IgG class, such as IgG1, IgG2, IgG3 and IgG4, can also be used. Also, as the CL of a human chimeric antibody, it may be any region which belongs to Ig, and those of kappa class or lambda class can be used. Methods for producing chimeric antibodies involve conventional recombinant DNA and gene transfection techniques are well known in the art (See Morrison S L. et al. (1984) and patent documents U.S. Pat. Nos. 5,202,238; and 5,204, 244).

In some embodiments, the monoclonal antibody of the invention is a humanized antibody. In particular, in said humanized antibody, the variable domain comprises human acceptor frameworks regions, and optionally human constant domain where present, and non-human donor CDRs, such as mouse CDRs. According to the invention, the term “humanized antibody” refers to an antibody having variable region framework and constant regions from a human antibody but retains the CDRs of a previous non-human antibody. The humanized antibody of the present invention may be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell. The humanized antibody expression vector may be either of a type in which a gene encoding an antibody heavy chain and a gene encoding an antibody light chain exists on separate vectors or of a type in which both genes exist on the same vector (tandem type). In respect of easiness of construction of a humanized antibody expression vector, easiness of introduction into animal cells, and balance between the expression levels of antibody H and L chains in animal cells, humanized antibody expression vector of the tandem type is preferred. Examples of tandem type humanized antibody expression vector include pKANTEX93 (WO 97/10354), pEE18 and the like. Methods for producing humanized antibodies based on conventional recombinant DNA and gene transfection techniques are well known in the art (See, e. g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No.5,565,332). The general recombinant DNA technology for preparation of such antibodies is also known (see European Patent Application EP 125023 and International Patent Application WO 96/02576).

In some embodiments the antibody of the invention is a human antibody. As used herein the term “human antibody is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. Human antibodies can be produced using various techniques known in the art. Human antibodies are described generally in van Dijk and van de Winkel, cur. Opin. Pharmacol. 5; 368-74 (2001) and lonberg, cur. Opin.Immunol. 20; 450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat.Biotech. 23;1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application publication No. US 2007/0061900, describing VELOCIMOUSE® technology. Human variable regions from intact antibodies generated by such animals may be further modified, e.g., by combining with a different human constant region Human antibodies can also be made by hybridoma-based methods. Human myeloma and mouse-human heteromyeloma cell lines for the production of human monoclonal antibodies have been described. (See, e.g., Kozbor J. Immunol., 13: 3001 (1984); Brodeur et al., Monoclonal Antibody Production Techniques and Applications, pp. 51-63 (Marcel Dekker, Inc., New York, 1987); and Boerner et al., J. Immunol., 147: 86(1991).) Human antibodies generated via human B-cell hybridoma technology are also described in Li et al., Proc. Natl. Acad. Sci. USA, 103:3557-3562 (2006). Additional methods include those described, for example, in U.S. Patent No. 7,189,826 (describing production of monoclonal human igM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005). Fully human antibodies can also be derived from phage-display libraries (as disclosed in Hoogenboom et al., 1991, J. Mol. Biol. 227:381; and Marks et al., 1991, J. Mol. Biol. 222:581). Phage display techniques mimic immune selection through the display of antibody repertoires on the surface of filamentous bacteriophage, and subsequent selection of phage by their binding to an antigen of choice. One such technique is described in PCT publication No. WO 99/10494. Human antibodies described herein can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In some embodiments, the antibody of the present invention mediates antibody-dependent cell-mediated cytotoxicity. As used herein the term “antibody-dependent cell-mediated cytotoxicity” or ‘ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. While not wishing to be limited to any particular mechanism of action, these cytotoxic cells that mediate ADCC generally express Fc receptors (FcRs).

As used herein “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgammal (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

The terms “Fc receptor” or “FcR” are used to describe a receptor that binds to the Fc region of an antibody. The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998). As used herein, the term Effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

In some embodiments, the antibody of the present invention is a full-length antibody. In some embodiments, the full-length antibody is an IgG1 antibody. In some embodiments, the full-length antibody is an IgG3 antibody.

In some embodiments, the antibody of the present invention comprises a variant Fc region that has an increased affinity for FcγRIA, FcγRIIA, FcγRIIB, FcγRIIIA, FcγRIIIB, and FcγRIV. In some embodiments, the antibody of the present invention comprises a variant Fc region comprising at least one amino acid substitution, insertion or deletion wherein said at least one amino acid residue substitution, insertion or deletion results in an increased affinity for FcγRIA, FcγRIIA, FcγRIIB, FcγRIIIA, FcγRIIIB, and FcγRIV, In some embodiments, the antibody of the present invention comprises a variant Fc region comprising at least one amino acid substitution, insertion or deletion wherein said at least one amino acid residue is selected from the group consisting of: residue 239, 330, and 332, wherein amino acid residues are numbered following the EU index. In some embodiments, the antibody of the present invention comprises a variant Fc region comprising at least one amino acid substitution wherein said at least one amino acid substitution is selected from the group consisting of: S239D, A330L, A330Y, and 1332E, wherein amino acid residues are numbered following the EU index.

In some embodiments, the glycosylation of the antibody is modified. For example, an aglycoslated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP 1 ,176,195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the human monoclonal antibodies of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al, 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17: 176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the human monoclonal antibodies of the present invention can be produced in yeasts or filamentous fungi engineered for mammalian- like glycosylation pattern and capable of producing antibodies lacking fucose as glycosylation pattern (see for example EP1297172B1

In some embodiments, the antibody of the present invention mediates complement dependant cytotoxicity. “Complement dependent cytotoxicity” or “CDC” refers to the ability of a molecule to initiate complement activation and lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), may be performed.

In some embodiments, the antibody of the present invention mediates antibody-dependent phagocytosis. As used herein, the term “antibody-dependent phagocytosis” or “opsonisation” refers to the cell-mediated reaction wherein nonspecific cytotoxic cells that express FcγRs recognize bound antibody on a target cell and subsequently cause phagocytosis of the target cell.

In some embodiments, the antibody of the present invention is a multispecific antibody comprising a first antigen binding site directed against CALCRL and at least one second antigen binding site directed against an effector cell as above described. In said embodiments, the second antigen-binding site is used for recruiting a killing mechanism such as, for example, by binding an antigen on a human effector cell. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs. In some embodiments, the second binding site binds to a Fc receptor as above defined. In some embodiments, the second binding site binds to a surface molecule of NK cells so that said cells can be activated.

Exemplary formats for the multispecific antibody molecules of the present invention include, but are not limited to (i) two antibodies cross-linked by chemical heteroconjugation, one with a specificity to a specific surface molecule of leukemic stem cell and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In : Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivaient bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the bispecific antibody is obtained or obtainable via a controlled

Fab-arm exchange, typically using DuoBody technology. In vitro methods for producing bispecific antibodies by controlled Fab-arm exchange have been described in WO2008119353 and WO 2011131746 (both by Genmab A/S). In one exemplary method, described in WO 2008119353, a bispecific antibody is formed by “Fab-arm” or “half- molecule” exchange (swapping of a heavy chain and attached light chain) between two monospecific antibodies, both comprising IgG4-like CH3 regions, upon incubation under reducing conditions. The resulting product is a bispecific antibody having two Fab arms which may comprise different sequences. In another exemplary method, described in WO 2011131746, bispecific antibodies of the present invention are prepared by a method comprising the following steps, wherein at least one of the first and second antibodies is a human monoclonal antibody of the present invention: a) providing a first antibody comprising an Fc region of an immunoglobulin, said Fc region comprising a first CH3 region; b) providing a second antibody comprising an Fc region of an immunoglobulin, said Fc region comprising a second CH3 region; wherein the sequences of said first and second CH3 regions are different and are such that the heterodimeric interaction between said first and second CH3 regions is stronger than each of the homodimeric interactions of said first and second CH3 regions; c) incubating said first antibody together with said second antibody under reducing conditions; and d) obtaining said bispecific antibody, wherein the first antibody is a human monoclonal antibody of the present invention and the second antibody has a different binding specificity, or vice versa. The reducing conditions may, for example, be provided by adding a reducing agent, e.g. selected from 2-mercaptoethylamine, dithiothreitol and tris(2-carboxyethyl)phosphine. Step d) may further comprise restoring the conditions to become non-reducing or less reducing, for example by removal of a reducing agent, e.g. by desalting. Preferably, the sequences of the first and second CH3 regions are different, comprising only a few, fairly conservative, asymmetrical mutations, such that the heterodimeric interaction between said first and second CH3 regions is stronger than each of the homodimeric interactions of said first and second CH3 regions. More details on these interactions and how they can be achieved are provided in WO 2011131746, which is hereby incorporated by reference in its entirety. The following are exemplary embodiments of combinations of such assymetrical mutations, optionally wherein one or both Fc-regions are of the IgG1 isotype. In some embodiments, the first Fc region has an amino acid substitution at a position selected from the group consisting of: 366, 368, 370, 399, 405, 407 and 409, and the second Fc region has an amino acid substitution at a position selected from the group consisting of: 366, 368, 370, 399, 405, 407 and 409, and wherein the first and second Fc regions are not substituted in the same positions. In some embodiments, the first Fc region has an amino acid substitution at position 405, and said second Fc region has an amino acid substitution at a position selected from the group consisting of: 366, 368, 370, 399, 407 and 409, optionally 409. In some embodiments, the first Fc region has an amino acid substitution at position 409, and said second Fc region has an amino acid substitution at a position selected from the group consisting of: 366, 368, 370, 399, 405, and 407, optionally 405 or 368. In some embodiments, both the first and second Fc regions are of the IgG1 isotype, with the first Fc region having a Leu at position 405, and the second Fc region having an Arg at position 409.

In some embodiments, the antibody of the present invention is conjugated to a therapeutic moiety, i.e. a drug. The therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody of the present invention is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin- inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, momordica charantia inhibitor, curcin, crotin, sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody of the present invention is conjugated to an auristatin or a peptide analog, derivative or prodrug thereof. Auristatins have been shown to interfere with microtubule dynamics, GTP hydrolysis and nuclear and cellular division (Woyke et al (2001) Antimicrob. Agents and Chemother. 45(12): 3580-3584) and have anti-cancer (US5663149) and antifungal activity (Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In some embodiments, the antibody of the present invention is conjugated to pyrrolo[2,1-c][1,4]- benzodiazepine (PDB) or an analog, derivative or prodrug thereof. Suitable PDBs and PDB derivatives, and related technologies are described in, e.g., Hartley J. A. et al., Cancer Res 2010; 70(17) : 6849-6858; Antonow D. et al., Cancer J 2008; 14(3) : 154-169; Howard P. W. et al., Bioorg Med Chem Lett 2009; 19: 6463-6466 and Sagnou et al., Bioorg Med Chem Lett 2000; 10(18) : 2083-2086.

In some embodiments, the antibody of the present invention is conjugated to a cytotoxic moiety selected from the group consisting of an anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In some embodiments, the antibody of the present invention is conjugated to an anthracycline or an analog, derivative or prodrug thereof. In some embodiments, the antibody is conjugated to maytansine or an analog, derivative or prodrug thereof. In some embodiments, the antibody is conjugated to calicheamicin or an analog, derivative or prodrug thereof. In some embodiments, the antibody is conjugated to duocarmycin or an analog, derivative or prodrug thereof. In some embodiments, the antibody is conjugated to rachelmycin (CC-1065) or an analog, derivative or prodrug thereof. In some embodiments, the antibody is conjugated to dolastatin 10 or an analog, derivative or prodrug thereof. In some embodiments, the antibody is conjugated to dolastatin 15 or an analog, derivative or prodrug thereof. In some embodiments, the antibody is conjugated to monomethyl auristatin E or an analog, derivative or prodrug thereof. In some embodiments, the antibody is conjugated to monomethyl auristatin F or an analog, derivative or prodrug thereof. In some embodiments, the antibody is conjugated to pyrrolo[2,1-c][1,4]-benzodiazepine or an analog, derivative or prodrug thereof. In some embodiments, the antibody is conjugated to irinotecan or an analog, derivative or prodrug thereof.

Techniques for conjugating molecule to antibodies, are well-known in the art (See, e.g., Amon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res.16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site- specifically conjugated to the Fc-containing polypeptide through the acyl donor glutamine- containing tag or the accessible/exposed/reactive endogenous glutamine (WO 2012059882).

In some embodiments, the inhibitor is a compound (e.g. an antibody) that inhibits the binding of CALCRL to RAMP1 and/or RAMP2 and/or RAMP3. In some embodiments, the inhibitor (e.g. an antibody) inhibits the binding of CALCRL to one of its ligand such as adrenomedullin.

In some embodiments, the inhibitor is an inhibitor of CALCRL, RAMP1, RAMP2, or RAMP3 expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of CALCRL, RAMP1, RAMP2, or RAMP3 mRNA by binding thereto and thus preventing protein translation or increasing mRNA degradation, thus decreasing the level of CALCRL, RAMP1, RAMP2, or RAMP3, and thus activity, in a cell. For example, antisense oligonucleotides of at least about 15 bases and complementary to unique regions of the mRNA transcript sequence encoding CALCRL, RAMP1, RAMP2, or RAMP3 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. CALCRL, RAMP1, RAMP2, or RAMP3 gene expression can be reduced by contacting a subject or cell with a small double stranded RNA (dsRNA), or a vector or construct causing the production of a small double stranded RNA, such that CALCRL, RAMP1, RAMP2, or RAMP3 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing CALCRL, RAMP1, RAMP2, or RAMP3. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Ban viruses; papilloma viruses; herpes virus; vaccinia virus; polio virus; and RNA virus such as a retrovirus. One can readily employ other vectors not named but known to the art.

By a “therapeutically effective amount” is meant a sufficient amount of the antibody or the inhibitor at a reasonable benefit/risk ratio applicable to the medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the antibody or inhibitor of the present invention is combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Sterile injectable solutions are prepared by incorporating the active ingredient at the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

A further object of the present invention relates to a method of identifying leukemic stem cells in a sample obtained from a subject suffering from AML comprising identifying and selecting the population of cells that expresses CALCRL and at least one marker of stem cell.

Samples for use in the diagnostic method of the present invention may be obtained from a variety of sources, particularly blood, although in some instances samples such as bone marrow, lymph, cerebrospinal fluid, synovial fluid, and the like may be used. Such samples can be separated by centrifugation, elutriation, density gradient separation, apheresis, affinity selection, panning, FACS, centrifugation with Hypaque, etc. prior to analysis. Once a sample is obtained, it can be used directly, frozen, or maintained in appropriate culture medium for short periods of time. Various media can be employed to maintain cells. The samples may be obtained by any convenient procedure, such as the drawing of blood, venipuncture, biopsy, or the like. Usually a sample will comprise at least about 10² cells, more usually at least about 10³ cells, and preferable 10⁴, 10⁵ or more cells. An appropriate solution may be used for dispersion or suspension of the cell sample. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

The leukemic stem cells can be prospectively isolated or identified from primary tumor samples. In particular leukemic stem cells possess the unique properties of cancer stem cells in functional assays for cancer stem cell self-renewal and differentiation. Methods for isolating leukemic cells and leukemic stem cells are well known in the art and typically involve the presence or absence of specific cell surface markers. For example, the comparison can be made between leukemic stem cells and the normal counterpart cells a human hematopoietic stem cell (HSC), which include without limitation cells having the phenotype Lin−CD34+CD38−CD90+; or the phenotype Lin−CD34+CD38−CD90+CD45RA− and a human hematopoietic multipotent progenitor cell (MPP), which include without limitation cells having the phenotype Lin−CD34+CD38−CD90−; or the phenotype Lin−CD34+CD38−CD90−CD45RA−.

In some embodiments, determining the presence or absence of the cell surface markers involves use of a panel of binding partners specific for the cell surface markers of interest. Said binding partners include but are not limited to antibodies, aptamer, and peptides. The binding partners will allow for the screening of cellular populations expressing the marker. Various techniques can be utilized to screen for cellular populations expressing the cell surface markers of interest, and typically include magnetic separation using antibody-coated magnetic beads, “panning” with antibody attached to a solid matrix (i.e., plate), and flow cytometry (See, e.g., U.S. Pat. No. 5,985,660; and Morrison et al. Cell, 96:737-49 (1999)).

In some embodiments, the binding partners are antibodies that may be polyclonal or monoclonal, preferably monoclonal, specifically directed against one cell surface marker. Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies of the invention or a fragment thereof can be prepared and isolated using any technique that provides for the production of antibody molecules by continuous cell lines in culture. Techniques for production and isolation include but are not limited to the hybridoma technique originally; the human B-cell hybridoma technique; and the EBV-hybridoma technique.

In some embodiments, the panel of binding partners is specific for at least one cell surface marker selected from the group consisting of CD33, CD34, CD36, CD38, CD39, CD45, CD81, CD90, and CD123 and thus comprise at least one binding partner specific for CALCRL.

Typically, the binding partners are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Typically each antibody is labeled with a different fluorochrome, to permit independent sorting for each marker. Suitable fluorescent detection elements include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 1996 Molecular Probes Handbook by Richard P. Haugland, hereby expressly incorporated by reference. Suitable fluorescent labels also include, but are not limited to, green fluorescent protein (GFP; Chalfie, et al., Science 263(5148):802-805 (Feb. 11, 1994); and EGFP; Clontech—Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), (β-galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. Nos. 5,292,658; 5,418,155; 5,683,888; 5,741,668; 5,777,079; 5,804,387; 5,874,304; 5,876,995; and 5,925,558). All of the above-cited references are expressly incorporated herein by reference. In some embodiments, detection elements for use in the present invention include: Alexa-Fluor dyes (an exemplary list including Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, AlexaFluor® 633, Alexa Fluor® 647, Alexa Fluor® 660, Alexa Fluor® 680, Alexa Fluor® 700, and Alexa Fluor® 750), Cascade Blue, Cascade Yellow and R-phycoerythrin (PE) (Molecular Probes) (Eugene, Oreg.), FITC, Rhodamine, and Texas Red (Pierce, Rockford, Ill.), Cy5, Cy5.5, Cy7 (Amersham Life Science, Pittsburgh, Pa.). Tandem conjugate protocols for Cy5PE, Cy5.5PE, Cy7PE, Cy5.5APC, Cy7APC are known in the art. Fluorophores bound to antibody or other binding element can be activated by a laser and re-emit light of a different wavelength. The amount of light detected from the fluorophores is related to the number of binding element targets associated with the cell passing through the beam. Any specific set of detection elements, e.g. fluorescently tagged antibodies, in any embodiment can depend on the types of cells to be studied and the presence of the activatable element within those cells. Several detection elements, e.g. fluorophore-conjugated antibodies, can be used simultaneously, so measurements made as one cell passes through the laser beam consist of scattered light intensities as well as light intensities from each of the fluorophores. Thus, the characterization of a single cell can consist of a set of measured light intensities that may be represented as a coordinate position in a multi-dimensional space. Considering only the light from the fluorophores, there is one coordinate axis corresponding to each of the detection elements, e.g. fluorescently tagged antibodies. The number of coordinate axes (the dimension of the space) is the number of fluorophores used. Modern flowcytometers can measure several colors associated with different fluorophores and thousands of cells per second. Thus, the data from one subject can be described by a collection of measurements related to the number of antigens for each of (typically) many thousands of individual cells. See Krutzik et al., High-content single-cell drug screening with phosphospecific flow cytometry. Nature Chemical Biology, Vol. 4 No. 2, Pgs. 132-42, February 2008. Such methods may optionally include the use of barcoding to increase throughput and reduce consumable consumption. See Krutzik, P. and Nolan, G., Fluorescent cell barcoding in flow cytometry allows high-throughput drug screening and signaling profiling. Nature Methods, Vol. 3 No. 5, Pgs. 361-68, May 2006.

In some embodiments the binding partner is conjugated to a metallic chemical element such as lanthanides. Lanthanides offer several advantages over other labels in that they are stable isotopes, there are a large number of them available, up to 100 or more distinct labels, they are relatively stable, and they are highly detectable and easily resolved between detection channels when detected using mass spectrometry. Lanthanide labels also offer a wide dynamic range of detection. Lanthanides exhibit high sensitivity, are insensitive to light and time, and are therefore very flexible and robust and can be utilized in numerous different settings. Lanthanides are a series of fifteen metallic chemical elements with atomic numbers 57-71. They are also referred to as rare earth elements. Lanthanides may be detected using CyTOF technology. CyTOF is inductively coupled plasma time-of-flight mass spectrometry (ICP-MS). CyTOF instruments are capable of analyzing up to 1000 cells per second for as many parameters as there are available stable isotope tags.

Typically, the binding partners are added to a suspension of cells, and incubated for a period of time sufficient to bind the available cell surface antigens. The incubation will usually be at least about 5 minutes and usually less than about 30 minutes. It is desirable to have a sufficient concentration of binding partners in the reaction mixture, such that the efficiency of the separation is not limited by lack of binding partners. The appropriate concentration is determined by titration. The medium in which the cells are separated will be any medium that maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The diagnostic method of the present invention is particularly suitable for determining whether the subject is at risk of relapse wherein the presence of said leukemic stem cells indicate that the subject is at risk of relapse. In some embodiments, the diagnostic method of the present invention is also particularly suitable for determine the survival time of the subject wherein the presence of said leukemic stem cells indicate that the subject will have a short survival time.

The invention will be further illustrated by the following figures and examples. However, these examples and figures should not be interpreted in any way as limiting the scope of the present invention.

### FIGURES:

FIG. 1: CALCRL downregulation impairs leukemic growth in vivo. A. Experimental plan for assessing leukemic stem cell frequency. After ex vivo treatment of cells with siCTR or siCALCRL, decreasing cell concentrations (500,000; 100,000; 10,000; 1000) were injected into the tail vein of mice (n=4 per group). Then, after 12 weeks, mice were dissected and human cell engraftment assessed in the murine bone marrow. Bonne marrow were considered as positively engrafted when the percentage of human cells was superior to 0.1%. B. Impact of CALCRL depletion on LIC frequency. C. Investigation of the role of CALCRL on leukemic cell growth in vivo. 2.10⁶ MOLM-14 or OCI-AML3 cells expressing doxycycline-inducible shCTR, shCAL#1 or shCAL#2 were injected into the tail vein of NSG mice. The day of injection, shRNA expression was induced adding 0.2mg/ml doxycycline in drinking water supplemented with 1% sucrose until the end of experiment. After 25 days, a part of mice were sacrificed to measure cell engraftment (total cell tumor burden=blasts number into bone marrow+spleen) (n=5-6 mice per group) and for another part of mice the survival parameter was followed (n=7-9 mice per group). D. Total cell tumor burden measured using mCD45.1−/hCD45+/hCD33+/AnnV-markers. Errors bars indicates Mean±SEM; groups were compared with unpaired two-tailed t test with Welch's correction. E. Mice survival monitoring. Groups were compared using log-rank (Mantel-Cox) test. *p<0.05; **p<0.01; *** p<0.001; ns, not significant.

FIG. 2: Depletion of CALCRL sensitizes cells to chemotherapy. A. Experimental plan for assessing the consequence of CALCRL depletion on chemotherapy response in vivo. 2.10⁶ MOLM-14 expressing indicated inducible shRNA were injected into the tail vein of NSG mice. Ten day after, when disease is established, mice were treated five days with 30mg/kg/d of cytarabine. B. Total cell tumor burden measured using mCD45.1-/hCD45+/hCD33+/AnnV-markers. C. Mice survival monitoring. Groups were compared using log-rank (Mantel-Cox) test. *p<0.05; **p<0.01; *** p<0.001; ns, not significant.

FIG. 3: Expression level of CALCRL predicts response to chemotherapy. A. Schematic diagram of the chemotherapy regimen and schedule used to treat NSG-based PDX models with AraC. Peripheral blood engraftment was assessed between 8 and 18 weeks, and mice were assigned to experimental groups of 4 to 10 mice with similar average engraftment per group. Mice were treated with vehicle (PBS) or 60 mg/kg/day AraC given daily via intraperitoneal injection for 5 days. Mice were sacrificed posttreatment at day 8 to characterize viable residual AML cells. B. Total number of human viable AML cells expressing hCD45, hCD33, and/or hCD44 were analyzed and quantified using flow cytometry in AraC-treated AML-xenografted mice compared with PBS-treated AML-xenografted mice in bone marrow. Fold reduction of total cell tumor burden in AraC-treated mice compared with control-treated mice was calculated individually for each AML patient sample. Patients were then spared into two categories: low responders (FC>10) and high responders (FC>10). C. Graphs shows the percentage of cells positive for CALCRL (cell surface expression was determined by flow cytometry analysis of vehicle-treated cells) in low vs high responder groups. D. Correlation between fold reduction and the percentage of CALCR-positive cells. Linear regression was performed to determine R² and p-value.*p<0.05; **p<0.01; *** p<0.001; ns, not significant.

FIG. 4: Targeting CALCRL eradicates chemoresistant leukemic stem cells. A. Role of CALCRL in the LSC-positive chemoresistant population. Primary AML sample was injected into blood of mice, then animals were treated with vehicle (PBS) or 60 mg/kg/day AraC given daily via intraperitoneal injection for 5 days. Mice were sacrificed posttreatment at day 8, and siRNA transfection was performed ex vivo on human cells from PBS and AraC treated conditions. Then decreasing cell concentrations were injected into the tail vein of mice (n=4 per group). After 12 weeks, mice were dissected and human cell engraftment was assessed in the murine bone marrow using mCD45.1-/hCD45+/AnnV-markers. Bonne marrows or spleens were considered as positively engrafted when the percentage of human cells was superior to 0.5%. B. Graph shows the LIC frequency into bone marrow and spleen. Frequency and statistics analyses were performed using L-calc software (Stemcell technologies).

### EXAMPLE:

Materiel and Methods:

Human Studies

Primary AML patient specimens are from Toulouse University Hospital (TUH), Toulouse, France]. Frozen samples were obtained from patients diagnosed with AML at TUH after signed informed consent in accordance with the Declaration of Helsinki, and stored at the HIMIP collection (BB-0033-00060). According to the French law, HIMIP biobank collection has been declared to the Ministry of Higher Education and Research (DC 2008-307, collection 1) and obtained a transfer agreement (AC 2008-129) after approbation by the Comité de Protection des Personnes Sud-Ouest et Outremer II (ethical committee). Clinical and biological annotations of the samples have been declared to the CNIL (Comite National Informatique et Libertes ie Data processing and Liberties National Committee). See Table S3 for age, sex, cytogenetics and mutation information on human specimens used in the current study.

In vivo animal studies

NSG (NOD.Cg-Prkdcscid I12rgtm1WjI/SzJ) mice (Charles River Laboratories) were used for transplantation of AML cell lines or primary AML samples. Male or Female mice ranging in age from 6 to 9 weeks were started on experiment and before cell injection or drug treatments, mice were randomly assigned to experimental groups. Mice were housed in sterile conditions using HEPA-filtered micro-isolators and fed with irradiated food and sterile water in the Animal core facility of the Cancer Research Center of Toulouse (France). All animals were used in accordance with a protocol reviewed and approved by the Institutional Animal Care and Use Committee of Région Midi-Pyrenees (France).

Cell Lines and Primary Cultures

For primary human AML cells, peripheral blood or bone marrow samples were frozen in FCS with 10% DMSO and stored in liquid nitrogen. The percentage of blasts was determined by flow cytometry and morphologic characteristics before purification. Cells were thawed in 37° C. water bath, washed in thawing media composed of IMDM, 20% FBS. Then cells were maintained in IMDM, 20% FBS and 1% Pen/Strep (GIBCO) for all experiments.

Cell Lines and Culture Conditions

Human AML cell lines were maintained in RPMI-media (Gibco) supplemented with 10% FBS (Invitrogen) in the presence of 100U/mL of penicillin and 100 μg/mL of streptomycin, and were incubated at 37° C. with 5% CO2. The cultured cells were split every 2 to 3 days and maintained in an exponential growth phase. All AML cell lines were purchased at DSMZ or ATCC, and their liquid nitrogen stock were renewed every 2 years. These cell lines have been routinely tested for Mycoplasma contamination in the laboratory. The U937 cells were obtained from the DSMZ in February 2012 and from the ATCC in January 2014. MV4-11 and HL-60 cells were obtained from the DSMZ in February 2012 and 2016. KG1 cells were obtained from the DSMZ in February 2012 and from the ATCC in March 2013. KG1 a cells were obtained from the DSMZ in February 2016. MOLM14 was obtained from Pr. Martin Carroll (University of Pennsylvania, Philadelphia, Pa.) in 2011.

Mouse Xenograft Model

NSG mice were produced at the Genotoul Anexplo platform at Toulouse (France) using breeders obtained from Charles River Laboratories. Transplanted mice were treated with antibiotic (Baytril) for the duration of the experiment. For experiments assessing the response to chemotherapy in PDX models, mice (6-9 weeks old) were sublethally treated with busulfan (30 mg/kg) 24 hours before injection of leukemic cells. Leukemia samples were thawed in 37° C. water bath, washed in IMDM 20% FBS, and suspended in Hank's Balanced Salt Solution at a final concentration of 1-10×106 cells per 200 μL for tail vein injection in NSG mice. Eight to 18 weeks after AML cell transplantation and when mice were engrafted (tested by flow cytometry on peripheral blood or bone marrow aspirates), NSG mice were treated by daily intraperitoneal injection of 60 mg/kg AraC or vehicle (PBS) for 5 days. AraC was kindly provided by the pharmacy of the TUH. Mice were sacrificed at day 8 to harvest human leukemic cells from murine bone marrow. For AML cell lines, 24 hours before injection of leukemic cells mice were treated with busulfan (20 mg/kg). Then cells were thawed and washed as previously described, suspended in HBSS at a final concentration of 2×106 per 200 μL before injection into bloodstream of NSG mice. For experiments using inducible shRNAs, doxycycline (0.2mg/ml+1% sucrose) was added to drinking water the day of cell injection or 10 days after until the end of the experiment. Mice were treated by daily intraperitoneal injection of 30 mg/kg AraC for 5 days and sacrificed at day 8. Daily monitoring of mice for symptoms of disease (ruffled coat, hunched back, weakness, and reduced mobility) determined the time of killing for injected animals with signs of distress.

Assessment of Leukemic Engraftment

At the end of experiment, NSG mice were humanely killed in accordance with European ethics protocols. Bone marrow (mixed from tibias and femurs) and spleen were dissected and flushed in HBSS with 1% FBS. MNCs from bone marrow, and spleen were labeled with anti-hCD33, anti-mCD45.1, anti-hCD45, anti-hCD3 and/or anti-hCD44 (all from BD) antibodies to determine the fraction of viable human blasts (hCD3-hCD45+mCD45.1−hCD33+/hCD44+AnnV-cells) using flow cytometry. In some experiments, we also added anti-CALCRL, anti-CD34 and anti-CD38 to characterize AML stem cells. Monoclonal antibody recognizing extracellular domain of CALCRL was generated in the lab with the help of Biotem company (France). Then antibody was labelled with R-Phycoerythrin using Lightning-Link kit (Expedeon). All antibodies were used at concentrations between 1/50 and 1/200 depending on specificity and cell density. Analyses were performed on a LSRFortessa flow cytometer with DIVA software (BD Biosciences) or CytoFLEX flow cytometer with CytoExpert software (Beckman Coulter). The number of AML cells/μL peripheral blood and number of AML cells in total cell tumor burden (in bone marrow and spleen) were determined by using CountBright beads (Invitrogen) using described protocol.

For LDA experiments, human engraftment was considered positive if at least >0.1% of cells in the murine bone marrow were hCD45+mCD45.1−hCD33+. The cut-off was increased to >0.5% for AML#31 because the engraftment was measured only based on hCD45+mCD45.1−. Limiting dilution analysis was performed using ELDA software.

Western Blot Analysis

Proteins were resolved using 4% to 12% polyacrylamide gel electrophoresis Bis-Tris gels (Life Technology, Carlsbad, CA) and electrotransferred to nitrocellulose membranes. After blocking in Tris-buffered saline (TBS) 0.1%, Tween 20%, 5% bovine serum albumin, membranes were immunostained overnight with appropriate primary antibodies followed by incubation with secondary antibodies conjugated to HRP. Immunoreactive bands were visualized by enhanced chemiluminescence (ECL Supersignal West Pico; Thermo Fisher Scientific) with a Syngene camera. Quantification of chemiluminescent signals was done with the GeneTools software from Syngene.

Cell Death Assay

After treatment, 5.105 cells were washed with PBS and resuspended in 200 μL of Annexin-V binding buffer (BD biosciences). Two microliters of Annexin-V-FITC (BD Biosciences) and 7-amino-actinomycin D (7-AAD; Sigma Aldrich) were added for 15 minutes at room temperature in the dark. All samples were analyzed using LSRFortessa or CytoFLEX flow cytometer.

Cell Cycle Analysis

Cells were harvested, washed with PBS and fixed in ice-cold 70% ethanol at −20° C. Cells were then permeabilized with 1× PBS containing 0.25% Triton X-100, resuspended in 1× PBS containing 10 μg/ml propidium iodide and 1 μg/ml RNase, and incubated for 30 min at 37° C. Data were collected on a CytoFLEX flow cytometer.

Clonogenic Essay

Primary cells from AML patients were thawed and resuspended in 100 μl Nucleofector Kit V (Amaxa, Cologne, Germany). Then, cells were nucleofected according to the manufacturer's instructions (program U-001 Amaxa, Cologne, Germany) with 200nM siRNA scrambled (ON-TARGETplus Non-targeting siRNA #2, Dharmacon) or anti-CALCRL (SMARTpool ON-TARGETplus CALCRL siRNA, Dharmacon). Cells were adjusted to 1×105 cells/ml final concentration in H4230 methylcellulose medium (STEMCELL Technologies) supplemented with 10% 5637-CM as a stimulant and then plated in 35-mm petri dishes in duplicate and allowed to grow for 7 days in a humidified CO2 incubator (5% CO2, 37° C.). At day 7, the leukemic colonies (more than five cells) were scored.

shRNA, lentiviral production and leukemic cell transduction

shRNA sequences were constructed into pLKO-TET-ON or bought cloned into pLKO vectors. Each construct (6 μg) was co-transfected using lipofectamine 2000 (20 μL) in 10 cm-dish with psPax2 (4 μg, provides packaging proteins) and pMD2.G (2 μg, provides VSV-g envelope protein) plasmids into 293T cells to produce lentiviral particles. Twenty-four hours after cell transfection, medium was removed and 10 ml opti-MEM+1% Pen/Strep was added. At about 72 hours post transfection, 293T culture supernatants containing lentiviral particles were harvested, filtered, aliquoted and stored in μ80° C. freezer for future use. At the day of transduction, cells were infected by mixing 2.106 cells in 2 ml of freshly thawed lentivirus and Polybrene at a final concentration of 8 ug/ml. At 3 days post infection, transduced cells were selected using 1μg/ml puromycin.

EC50 Experiments

The day before experiment, cells were adjusted to 3×105 cells/ml final concentration and plated in a 96-well plate (final volume: 100 μl). To measure half-maximal inhibitory concentration (EC50), increased concentrations of AraC or idarubicin were added to the medium. After two days, 20 μl per well of MTS solution (Promega) was added for two hours and then absorbance was recorded at 490 nm with a 96-well plate reader. The doses that decrease cell viability to 50% (EC50) were analyzed Nonlinear regression log [inhibitor] vs. normalized response-variable slope with GraphPad Prism software.

Measurement of Oxygen Consumption in AML Cultured Cells using Seahorse Assay

All XF assays were performed using the XFp Extracellular Flux Analyser (Seahorse Bioscience, North Billerica, Mass.). The day before the assay, the sensor cartridge was placed into the calibration buffer medium supplied by Seahorse Biosciences to hydrate overnight. Seahorse XFp microplates wells were coated with 50 μl of Cell-Tak (Corning; Cat#354240) solution at a concentration of 22.4 μg/ml and kept at 4° C. overnight. Then, Cell-Talc coated Seahorse microplates wells were rinsed with distillated water and AML cells were plated at a density of 105 cells per well with XF base minimal DMEM media containing 11 mM glucose, 1 mM pyruvate and 2 mM glutamine. Then, 180 μl of XF base minimal DMEM medium was placed into each well and the micrcoplate was centrifuged at 80 g for 5 min. After one hour incubation at 37° C. in CO2 free-atmosphere, basal oxygen consumption rate (OCR, as a mitochondrial respiration indicator) and extracellular acidification rate (ECAR, as a glycolysis indicator) were performed using the XFp analyzer.

RNA Microarray and Bioinformatics Analyses

For primary AML samples, human CD45+ CD33+ were isolated using cell sorter cytometer from engrafted BM mice (for 3 primary AML specimens) treated with PBS or treated with AraC. RNA from AML cells was extracted using Trizol (Invitrogen) or RNeasy (Qiagen). For MOLM-14 AML cell line, mRNA from 2.106 of cells was extracted using RNeasy (Qiagen). RNA purity was monitored with NanoDrop 1ND-1000 spectrophotometer and RNA quality was assessed through Agilent 2100 Bionalyzer with RNA 6000 Nano assay kit. No RNA degradation or contamination were detected (RIN >9). 100 ng of total RNA were analysed on Affymetrix GeneChip© Human Gene 2.0 ST Array using the Affymetrix GeneChip© WT Plus Reagent Kit according to the manufacturer's instructions (Manual Target Preparation for GeneChip® Whole Transcript (WT) Expression Arrays P/N 703174 Rev. 2). Arrays were washed and scanned; and the raw files generated by the scanner was transferred into R software for preprocessing (with RMA function, Oligo package), quality control (boxplot, clustering and PCA) and differential expression analysis (with eBayes function, LIMMA package). Prior to differential expression analysis, all transcript clusters without any gene association were removed. Mapping between transcript clusters and genes were done using annotation provided by Affymetrix (HuGene-2_0-st-v1.na36.hg19.transcript.csv) and the R/Bioconductor package hugene20sttranscriptcluster.db. p-values generated by the eBayes function were adjusted to control false discovery using the Benajmin and Hochberg's procedure. [RMA] Irizarry et al., Biostatistics, 2003; [Oligo package] Carvalho and Irizarry, Bioinformatics, 2010; [LIMMA reference] Ritchie et al., Nucleic Acids Research, 2015; hugene20sttranscriptcluster.db :MacDonald JW 2017, Affymetrix hugene20 annotation data (chip hugene20sttranscriptcluster); [FDR]: Benjamini et al., Journal of the Royal Statistical Society, 1995.

GSEA Analysis

GSEA analysis was performed using GSEA version 3.0 (Broad Institute). Gene signatures used in this study were from Broad Institute database, literature, or in-house built. Following parameters were used: Number of permutations=1000, permutation type=gene_set. Other parameters were left at default values.

Quantification and Statistical Analysis

We assessed the statistical analysis of the difference between two sets of data using two-tailed (non-directional) Student's t test with Welch's correction. For survival analyses, we used Log-rank (Mantel-Cox) test. For Limit Dilution Assay experiments, frequency and statistics analyses were performed using L-calc software (Stemcell technologies). A p value of less than 0.05 indicates significance. *p<0.05; **p<0.01; ***p<0.001; ****p<0.0001; ns, not significant. Detailed information of each test is in the figure legends.

Results

Leukemic Stem Cells have Higher CAL CRL Expression Required for their Maintenance

Using a clinically relevant chemotherapeutic model, we previously demonstrated that LSCs are not necessarily enriched in AraC residual AML, suggesting that these cells are also targeted by chemotherapy and the existence of both chemosensitive and chemoresistant stem cell sub-populations (Farge et al., 2017). In order to identify drug-resistant LSCs, we analyzed transcriptomic data from three different studies: i) that identified 134 genes overexpressed in functionally defined LSCs compared with normal HSCs counterpart (Eppert et al., 2011), ii) that uncovered 114 genes which high expression is associated with poor prognosis in AML (the Cancer Genome Atlas, AML cohort, 2013) and iii) that selected 536 genes overexpressed at relapse compared to pairwise matched diagnosis after intensive chemotherapy (Hackl et al., 2015). Surprisingly, we found one unique gene common to these three transcriptomic databases: CALCRL encoding for a G Protein-Coupled Receptor and not yet described in cancer and AML. Using three independently published cohorts of AML patients (TCGA, AML cohort; GSE12417; GSE14468), we showed that patients with high CALCRL expression significantly had low overall survival and event free survival. Furthermore, CALCRL gene expression was higher in AML cells of patients at relapse compared to their matched cells at diagnosis, and in the leukemic compartment compared with normal, and more specifically in the LSC population as both functionally-and phenotypically- defined. Using a monoclonal antibody developed by our laboratory, we confirmed by flow cytometry analysis that cell surface expression of CALCRL is significantly upper in leukemic bulk (n=37) vs. normal bulk (n=9) (Fold change, FC=2.3) and enriched in the immature CD34⁺ CD38⁻ compartment of AML patients (FC AML CD34⁺ CD38⁻ vs AML bulk=1.4; FC AML CD34⁺ CD38⁻ vs normal CD34⁺CD38⁻=2.6, respectively). Interestingly, the percentage of positive cells for the receptor is similar in all studied populations, indicating an overexpression of CALCRL in a minority population of AML cells. Then, we performed similar approach to assess the expression of ADM, a CALCRL ligand already described in several cancer models. The results showed that ADM gene is overexpressed in AML cells compared to normal cells and its gene expression is not modified at the relapse of post-chemotherapy in AML patients. Combining RNA microarray, confocal microscopy and western blotting studies, we have established that CALCRL, its three co-receptors RAMP1, RAMP2 and RAMP3, ADM (but not the CGRP, another putative ligand) are expressed in all tested cell lines and that the CALCRL receptor is well present at the plasma membrane of these cells. These observations were also confirmed in primary AML patient samples. Thus, cellular expression of CALCRL in immature AML cells suggested a new marker of LSC and a putative role of this receptor in LSC biology.

Then we addressed the impact of CALCRL and ADM protein level on patient outcome. Using IHC analyses, we observed that increasing protein levels of CALCRL or ADM were associated with decreasing complete remission rates, 5-year overall, and event-free survival in a cohort of 198 AML patients. When patients are clustered into 4 groups according to CALCRL and ADM expression (−/− vs −/+vs +/− vs +/+; data not shown), we observed that the CALCRLhigh/ADMhigh group had a strong reduction of overall survival and that the high expression of only CALCRL or ADM is sufficient to dramatically reduce EFS and complete remission rate (data not shown). All these data supported the hypothesis that the ADM-CALCRL axis is activated in an autocrine fashion in AML and related to a poor prognosis.

CALCRL is Required for Leukemic Stem Cell Maintenance

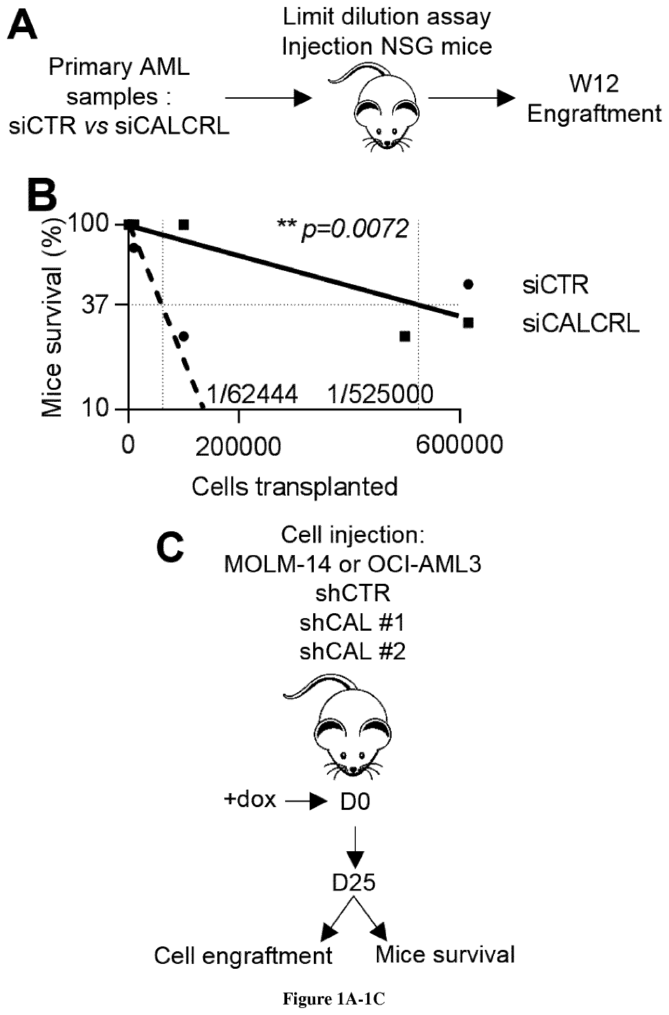

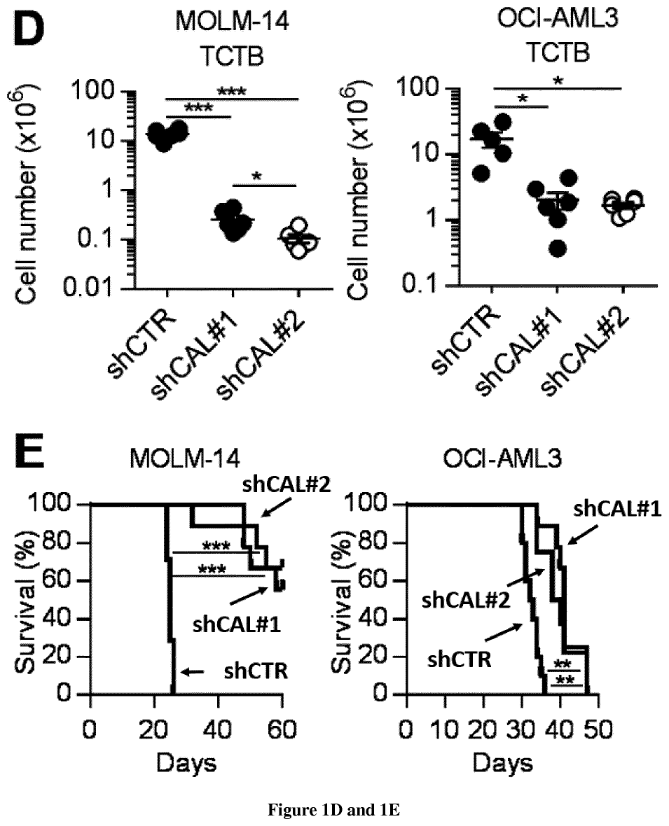

Using Gene Seat Enrichment Analysis (GSEA) approaches, we first confirmed that several LSC-associated gene signatures (Eppert et al., 2011; Gentles, 2010; Ng et al., 2016) are enriched in AML patients with the highest CALCRL expression compared to AML patients with the lowest CALCRL expression. In order to specifically study the role of CALCRL in the maintenance of LSC function, we first performed ex vivo assays knocking down CALCRL in a primary AML sample followed by injection of different doses (limiting dilution analysis) of cells in NSG immunocompromised mice (FIG. 1A). After 12 weeks, the mice were sacrificed and the percentage of human cells in the bone marrow of the animals was evaluated. The results showed that invalidating CALCRL significantly decreased the frequency of LSCs in vivo in secondary transplantations (1/62,444 in siCTR vs 1/525,000 in siCALCRL, FIG. 1B), demonstrating its requirement in preserving the function of LSCs.

CAL CRL is Required for Cell Growth and Survival In Vitro and In Vivo

Since the role of several GPCRs identified in AML such as CXCR4 or GPR56 on cell survival and proliferation (Chen et al., 2013), we then investigated whether the ADM-CALCRL axis could have an impact on these features. CALCRL was knock-downed by shRNA in MOLM-14 and OCI-AML3 cell lines. We have shown that CALCRL depletion is associated with a decrease of blast cell proliferation, an increase of cell death and a cleavage of pro-apoptotic markers caspase-3 and PARP. Furthermore, adrenomedullin-targeting shRNA phenocopied the effects of shCALCRL on cell proliferation and apoptosis in MOLM-14 and OCI-AML3 cells. In order to confirm these results in vivo and to maintain the invalidation of the target over time, we have developed tetracycline-inducible shRNA models. First, we confirmed that the depletion of CALCRL was associated with a decrease in cell proliferation and an increase in apoptosis as with constitutive shRNAs approaches. After injection of AML cells in mice, RNA depletion was activated from the first day by doxycycline (FIG. 1C). Twenty-five days post-transplantation, the engraftment of human leukemic cells from murine bone marrow and spleen was assessed with mCD45.1⁻hCD45⁺hCD33⁺AnV⁻ markers (FIG. 1C). We observed that mice injected with shCAL#1 and shCAL#2 had significantly less AML blasts than shCTR both for MOLM-14 (shCTR=13.9M vs shCAL#1=0.3M vs shCAL#2=0.1M, FIG. 1D) and for OCI-AML3 cells (shCTR=17.2M vs shCAL#1=2.0M vs shCAL#2=1.7M, FIG. 1D). Accordingly, this reduction in total cell tumor burden led to a preservation of the murine bone marrow. Finally, the CALCRL knock-down significantly extended mice survival (FIG. 1E). In order to take full advantage of our inducible model and to improve the clinical relevance of our model, we assessed the impact of CALCRL depletion on an established disease. shRNA expression was induced 10 days post-transplantation of shCTR or shCAL MOLM14 cells, after verifying that the level of engraftment was similar in both groups and group randomization. In this established disease model, we observed a marked reduction in bone marrow blasts in the mice xenografted with shCAL AML cells compared to the shCTR mice cohort, for which the disease progressed. Furthermore, CALCRL downregulation induced a significant increase in mice survival. Altogether, these results demonstrated that CALCRL was required for the propagation but also for the maintenance of AML cells in vivo.

Depletion of CALCRL Decreases Cellular Energetic Status, BCL2, Cell Cycle and DNA Repair Pathways in AML

To determine regulatory pathways downstream of CALCRL, we have generated and performed comparative transcriptomic and functional assays on shCTR vs shCALCRL MOLM-14 cells were generated. Interestingly, the knockdown of CALCRL is associated with a significant decrease in the expression of 623 genes and an increase of 278 (FDR<0.05, p-value<0.05). As mitochondrial metabolism emerges as a critical regulator of cell proliferation and survival in AML (Scotland et al., 2013; Skrtic et al., 2015; Molina et al., 2018), we first analyzed the impact of CALCRL depletion on this pathway. GSEA showed a significant depletion in the gene signatures associated with mitochondrial oxidative metabolism in the shCALCRL MOLM-14 cells. Moreover, OCR measurements revealed a reduction in basal OCR, whereas maximal respiration was conserved and spare capacity increased, suggesting that cells might able to enhance mitochondrial use if needed. Consistently we also observed a significant decrease of mitochondrial ATP production and a downregulation of the mitochondrial transcription factor TFAM. Finally, basal cellular energetic status is decreased in the absence of CALCRL, suggesting a shift from a proliferative state to a quiescent state of shCALCRL cells. Consistently with this hypothesis, datamining analyses have shown significant enrichment in genes involved in cell cycle and DNA integrity pathways in the shCTR cells. Western blotting confirmed that CALCRL depletion affected the expression of RAD51 and CHEK1 and BCL2 protein levels. This was associated with an accumulation of cells in the Go/Gi phase. Interestingly, enrichment analysis showed that depletion of CALCRL affects the gene signatures of several key transcription factors such as E2F1, P53 or FOXM1 largely described as critical cell cycle regulators.

We next focused on E2F1 transcription factor, whose importance in the biology of LSCs from chronic myeloid leukemia has recently been discussed (Pellicano et al., 2018). We first confirmed that the depletion of CALCRL is well associated with a significant decrease in the activity of E2F1. Then we demonstrated that the knockdown of E2F1 affected the protein expression of RAD51, CHK1 but not BCL2, inhibited cell proliferation, cell cycle progression and induced high cell death in both MOLM-14 and OCI-AML3. In addition, the depletion of E2F1 affected cellular and mitochondrial energetic status. These results strongly strengthen that E2F1 is well downstream of CALCRL and governs cell proliferation, survival and metabolism. Then we investigated if CALCRL regulated the proliferation of primary AML cells. Interestingly we first observed that the protein level of CALCRL was positively correlated with clonogenic capacities in methylcellulose. As expected the depletion of CALCRL in primary samples decreased the number of colonies and the protein levels of BCL2 and RAD51. All these results suggested that CALCRL had a role in the proliferation of AML blasts and controls critical pathways involved in DNA repair processes.

CALCRL Downregulation Sensitizes Leukemic Cells to Chemotherapeutic Drugs Cytarabine and Idarubicin

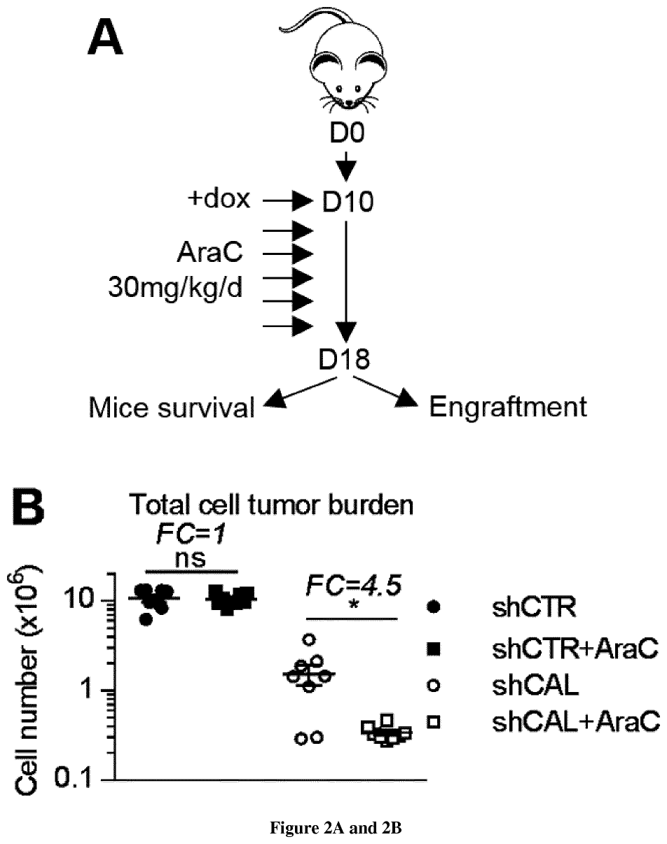

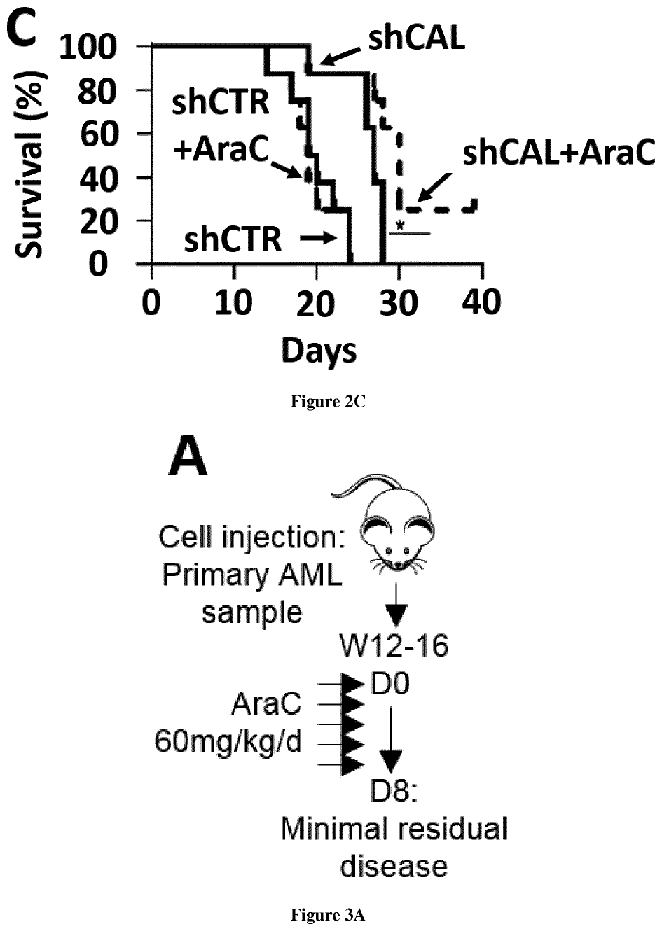

Considering proteins positively regulated by CALCRL, such as BCL2, CHK1, or FOXM1 (David et al., 2016; Khan et al., 2017; Konopleva et al., 2016), we hypothesized that CALCRL was involved in chemoresistance processes. Thus, the depletion of CALCRL sensitized MOLM-14 and OCI-AML3 cells to cytarabine and idarubicin, whether in terms of cell viability, induction of cell death and increased cleavage of apoptotic proteins CASPASE-3 and PARP. Furthermore, we have also shown that the depletion of ADM or E2F1 also sensitized cells to compounds. This demonstrated that the ADM-CALCRL-E2F1 axis played a role in drug resistance in vitro. In order to confirm these results in vivo, we used our model of inducible shRNA targeting CALCRL. After validating in vitro that these inducible shRNAs recapitulated well the sensitization observed with constitutive shRNAs, MOLM-14 cells transduced with shCTR or shCAL#2 were injected into the blood of NSG mice. After 10 days, shRNA expression was activated and the mice were treated with 30 mg/kg/day cytarabine for 5 days (FIG. 2A). We were able to show that AraC, in combination with shCALCRL, significantly reduced the number of total blasts compared with shCTR+/− AraC and shCALCRL alone (FIG. 2B), induced higher rates of cell death, and prolonged mice survival (FIG. 2C). Also MOLM-14 expressing shCTR and treated by vehicle or AraC were sorted by flow cytometry, and plated in vitro for further experiments. Interestingly, after one week of in vitro culture, the cells from AraC treated mice were more resistant to AraC (EC50: 2.238 μM for vehicle group vs 6.712 μM for AraC treated group) and idarubicin (EC50: 28.64nM for vehicle group vs 60.55 nM for AraC treated group). These results strongly suggested resistance mechanisms common to both drugs that persisted over time. Next, we showed that cells that were treated with AraC in vivo had higher protein expression levels of CALCRL, but also a slight increase in RAD51 and BCL2, whereas CHK1 was similar to untreated. To evaluate the role of CALCRL in this chemoresistance acquired in vivo, we undertook to deplete CALCRL in these cells. Thus, the knockdown of CALCRL by two different shRNAs sensitized cells to AraC and idarubicin, whether cells treated by the vehicle or by AraC. Remarkably, the EC50 of AraC and idarucibin in AraC-treated cells in vivo and transduced with shCALCRL #1 and #2 were similar to those observed for MOLM-14 from Vehicle-treated mice and then transduced with shCTR. This suggested hardly that the increase of CALCRL and downstream signaling pathways partly but not completely explained the chemoresistance of cells. Altogether, all of these results indicate robustly that CALCRL drives chemoresistance in AML cells.

CALCRL-Dependent Expression of BCL2 is Required to Maintain High Oxphos Status and Resistance to Chemotherapeutic Drugs

We have previously proposed that after AraC (cytarabine) treatment, residual cells have exacerbated oxidative metabolism and that targeting mitochondria in combination with conventional chemotherapy may be an innovative therapeutic approach in AML (Farge et al., 2017). Since the depletion of CALCRL in our cells decreased the oxidative metabolism, we assessed the cellular energetic status in association with AraC. Thus, the knockdown of CALCRL significantly counteracted AraC-induced increase of basal respiration, maximal respiration or reserve capacities, in response to AraC. Moreover we also observed a decrease in mitochondrial ATP production in response to AraC whereas ECAR was not affected. This result had put us on the way of BCL2, whose roles in drug resistance and in controlling the oxidative status of AML cells have already been described. First we showed that the overexpression of BCL2 in MOLM-14 did not modify OCR, mitochondrial ATP production and ECAR, suggesting that basal levels of BCL2 were not necessary involved in the control of mitochondrial energetic status. However, in response to AraC treatment, we observed that overexpression of BCL2, is sufficient to rescue maximal respiration and spare capacities similar reserve but not basal respiration. This would therefore indicate a role of the CALCRL-BCL2 axis to maintain the ability of mitochondria to respond to AraC. This was not related to energy production as nor mitochondrial ATP production nor ECAR were affected. Finally, BCL2 overexpression completely inhibited basal apoptosis induced by the depletion of CALCRL and by the combination with AraC or idarubicin.

Chemotherapy Selects CALCRL-Positive Leukemic Stem Cells

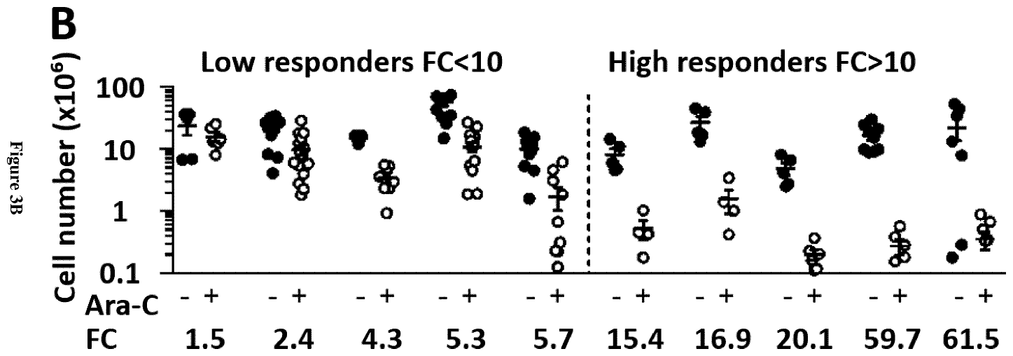

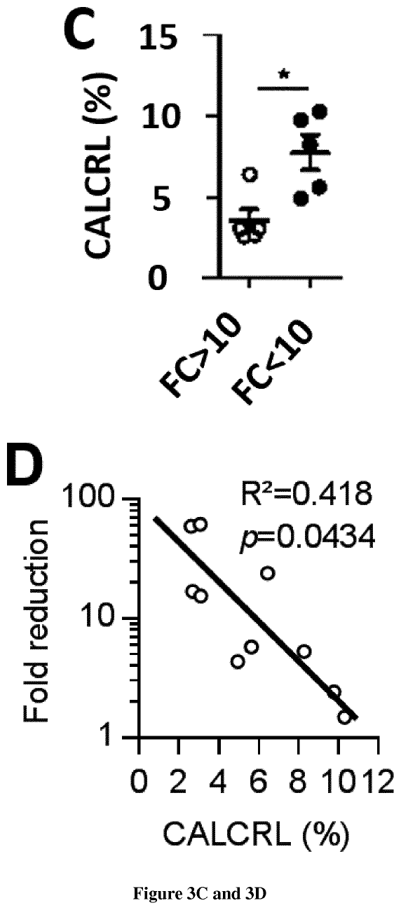

We used a clinically relevant PDX model of AraC-treated mice to address the role of CALCRL in response to chemotherapy in primary AML samples (Farge et al., 2017). After injection of primary cells into the blood of NSG mice, when engraftment was confirmed, mice were treated for 5 days with 60 mg/kg/day of AraC and sacrificed at day 8 to study the minimal residual disease (FIG. 3A). We tested 10 different PDXs and ranked them according to their response to AraC as low (FC Vehicle/AraC <10) or high (FC>10) responders (FIG. 3B). The percentage of cells positive for CALCRL was twice as high in the low responder group (3.578% vs 7.786%) and we observed an inverse linear correlation between the percentage of positive cells and the tumor reduction (R²=0.418) (FIGS. 3C and 3D). Moreover, in cells from minimal residual disease, a significant increase in the percentage of positive blasts for CALCRL (5.6% vs 23%) was observed whatever the studied sub-population. On the contrary, the mean fluorescence intensity of CALCRL-positive cells remained generally constant. These results would suggest that chemotherapy selected CALCRL-positive cells rather to increase CALCRL expression levels in blasts. To further investigate the role of CALCRL in response to chemotherapy in the context of LSCs, we used an approach combining single-cell RNA-seq (scRNA-seq) assays at diagnosis and relapse, after injection into Immunocompromised mice as well as the determination of stem cell frequency of CALCRL+ vs CALCRL− cells. First, scRNA-seq analysis at the diagnosis revealed two cell clusters associated with different gene expression signatures. After injection into mice, only the cluster 1 was present, meaning that only cells with this gene signature were able to engraft in NSG mice. In addition, GSEA analyses have shown that this cluster is enriched in CALCRL_UP (previously defined in our transcriptomes) and LSC+_UP (functionally defined by Eppert et al.) genes. At relapse, only cluster 1′ (related to cluster 1) was present and enriched in CALCRL_UP, LSC+_UP and Relapse_UP (Hackl et al.) genes. Finally, LDA studies carried out by sorting CALCRL− and CALCRL+ cell populations at diagnosis and relapse followed by injection into mice showed that the CALCRL+ cell population was significantly enriched in stem cells at diagnosis compared with CALCRL− population. At relapse, we observed a global increase in LSC frequency in the CALCRL− and CALCRL+ compartments, suggesting the acquisition of a stem cell phenotype by both populations.

Recently Shlush et al. proposed an elegant model of relapses with two situations: in the first one called “relapse origin - primitive” (ROp), relapse originated from rare LSC clones only detectable in HSPC or after xenotransplantation. In the second model, called “relapse origin-committed” (ROc), relapse clone arose from immunophenotypically committed leukemia cells in which bulk cells harbored a stemness transcriptional profile (Shlush et al., 2016). We analyzed this transcriptomic database and observed that at the time of diagnosis, CALCRL expression was higher in blasts with ROc than with ROp phenotype, in accordance with the expression of CALCRL in cells harboring stem cell features (Data not shown). Interestingly, CALCRL was strongly increased at relapse in ROp patients, which correlated with the emergence at this stage of the disease of a clone with stem cell properties (Data not shown). These observations supported our hypothesis of the preexistence of a relapse-relevant LSC population, rare (ROp) or abundant (ROc), expressing high levels of CALCRL.

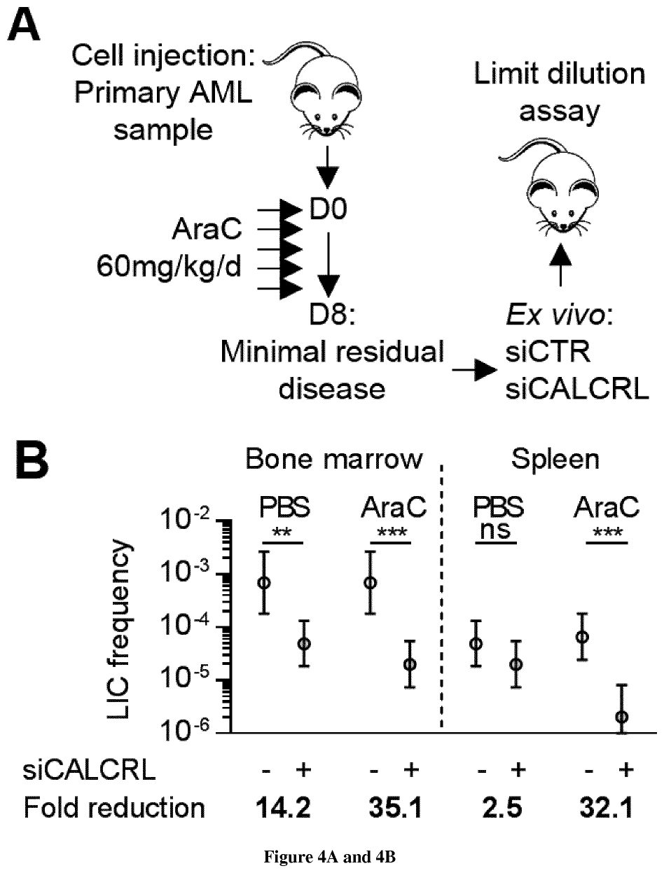

Another issue was to determine the role of CALCRL in the chemoresistance of LSCs. Primary AML cells were injected into NSG mice, and after engraftment and treatment with AraC the human cells were sorted and then transfected with siCTR or siCALCRL before reinjection into the mice to determine the frequency of stem cells (FIG. 4A). Thus, it was observed both in the bone marrow and the spleen, a significant reduction of stem cell frequency in the siCALCRL condition compared to the siCTR condition (FIG. 4B). In addition, fold reduction was superior in AraC-treated conditions compared to vehicle-treated conditions, suggesting a greater dependence of residual chemoresistant stem cells to CALCRL. Altogether, all of these results strongly support the hypothesis that chemotherapy selects a cell population enriched in LSC and positive for CALCRL.

Discussion:

Clinical efficacy of LSC-selective targeted therapies has not been proven for AML treatment due to high plasticity and heterogeneity not only for the phenotype (Taussig et al., 2010; Eppert et al., 2011; Sarry et al., 2011) but also for the drug sensitivity (Farge et al., 2017; Boyd et al., 2018) of the LSC population. However, fundamental studies focusing on intrinsic properties of this cell population such as their resistance to chemotherapy are crucially needed for the development of improved and more specific therapies in AML.

Our study provides key insights of LSC biology and drug resistance and identifies the ADM receptor CALCRL as a master regulator of R-LSCs. Our work first shows that CALCRL gene is overexpressed in the leukemic compartment compared to normal counterpart based on Eppert' s study that functionally characterizes LSCs. CALCRL could be specifically upregulated by LSC-related transcription factors such as HIFI a or ATF4 (Wang et al., 2011; van Galen et al., 2018). Indeed, both ADM and CALCRL possess the consensus hypoxia-response element (HRE) in the 5′-flanking region and are HIF 1 a-regulated genes (Nikitenko et al., 2003). Recently, it has been demonstrated that the integrated stress response and the transcription factor ATF4 is involved in AML cell proliferation and is uniquely active in HSCs and LSCs (van Galen et al., 2018; Heydt et al., 2018). Interestingly, maintenance of murine HSCs under proliferative stress but not steady-state conditions is dependent on CALCRL signaling (Suekane et al., 2019). Accordingly, CALCRL might support leukemic hematopoiesis and overcome stress induced by the high proliferation rate of AML cells.

Our findings clearly show that targeting CALCRL expression impacts clonogenic capacities, cell cycle progression and genes related to DNA repair and genomic stability. If cancer stem cells and LSCs are predominantly quiescent thereby preserving them from chemotherapy, recent studies suggested that LSCs also display a more active cycling phenotype (Iwasaki et al., 2015; Pei et al., 2018). C-type lectin CD93 is expressed on a subset of actively cycling, non-quiescent AML cells enriched for LSC activity (Iwasaki et al., 2015). Recently, Pei et al. showed that targeting the AMPK-FIS1 axis disrupted mitophagy and induced cell cycle arrest in AML, leading to the depletion of LSC potential in primary AML. These results are consistent with the existence of different sub-populations of LSCs that differ in proliferative state. Moreover, FIS1 depletion induces the down-regulation of several genes (e.g. CCND2, CDC25A, PLK1, CENPO, AURKB) and of the E2F1 gene signature that both we also identified after CALCRL knockdown. Recently, it has been proposed that E2F1 plays a pivotal role in regulating CML stem/progenitor cells proliferation and survival status (Pellicano et al., 2018). Several signaling pathways, for instance MAPKs, CDK/cyclin or PI3K/AKT, have been described to be stimulated by ADM/CALCRL axis and may control pRB/E2F1 complex activity (Hallstrom et al., 2008; Wang et al., 1998). Other signaling mediators activated in LSCs such as c-Myc and CEBPα regulate E2F1 transcription and allow the interaction of the E2F1 protein with the E2F gene promoters to activate genes essential for DNA replication at G 1/S, cell proliferation and survival in AML (Leung et al., 2008; O′Donnell et al., 2005; Rishi et al., 2014). Therefore, this analysis of cellular signaling downstream of CALCRL uncovers new pathways crucial for the maintenance and the chemoresistance of LSCs.

The characterization of chemotherapy-spared R-LSCs, which are present at the onset of relapse, is necessary to develop new therapeutic strategies to eradicate them. Boyd and colleagues have proposed the existence of a transient state of Leukemic Regenerating Cells (LRC) during the immediate and acute response to AraC that are responsible for disease regrowth foregoing the recovery of LSC pool (Boyd et al., 2018). In this attractive model and in the dynamic of MRD post-chemotherapy, CALCRL-positive AML cells are a part of this LRC subpopulation and CALCRL is essential for the preservation of LSC potential of chemoresistant primary AML. It would be interesting to determine whether chemotherapy only spares cells that are positive for CALCRL and/or whether it induces an adaptive response to stress that increases the expression of CALCRL. Transcription factors that are activated in response to chemotherapy should be identified to improve our knowledge of the acute response to chemotherapy. Therefore, therapeutic targeting of CALCRL should be clinically investigated to specifically eradicate MRD and prevent relapse in AML. Finally, as several molecules preventing the binding of the neuropeptide CGRP to CALCRL have been developed for treatment in other diseases (Hutchings et al., 2017; Schuster and Rapoport, 2017), this facilitates future pharmacological approaches to antagonize ADM-CALCRL axis in AML.

In summary, our data clearly identify CALCRL as a new stem cell actor required to sustain AML development in vivo. This receptor regulates genes involved in chemoresistance mechanisms and its depletion sensitizes AML cells to both cytarabine and anthracyclines in vitro and in vivo. This further indicates that LSCs resistant to these drugs share common activated pathways involved in these resistance mechanisms. All of these results strongly suggest CALCRL is a new and promising candidate therapeutic target for anti-LSC therapy.

### REFERENCES

Throughout this application, various references describe the state of the art to which this invention pertains. The disclosures of these references are hereby incorporated by reference into the present disclosure.

Boyd, A. L., Aslostovar, L., Reid, J., Ye, W., Tanasijevic, B., Porras, D. P., Shapovalova, Z., Almakadi, M., Foley, R., Leber, B., et al. (2018). Identification of Chemotherapy-Induced Leukemic-Regenerating Cells Reveals a Transient Vulnerability of Human AML Recurrence. Cancer Cell 34, 483-498.e5.

Chen, Y., Jacamo, R., Konopleva, M., Garzon, R., Croce, C., and Andreeff, M. (2013). CXCR4 downregulation of let-7a drives chemoresistance in acute myeloid leukemia. J. Clin. Invest. 123, 2395-2407.

David, L., Fernandez-Vidal, A., Bertoli, S., Grgurevic, S., Lepage, B., Deshaies, D., Prade, N., Cartel, M., Larrue, C., Sarry, J.-E., et al. (2016). CHK1 as a therapeutic target to bypass chemoresistance in AML. Sci. Signal. 9, ra90-ra90.

Eppert, K., Takenaka, K., Lechman, E. R., Waldron, L., Nilsson, B., van Galen, P., Metzeler, K. H., Poeppl, A., Ling, V., Beyene, J., et al. (2011). Stem cell gene expression programs influence clinical outcome in human leukemia. Nat. Med. 17, 1086-1093.

Farge, T., Saland, E., de Toni, F., Aroua, N., Hosseini, M., Perry, R., Bosc, C., Sugita, M., Stuani, L., Fraisse, M., et al. (2017). Chemotherapy-Resistant Human Acute Myeloid Leukemia Cells Are Not Enriched for Leukemic Stem Cells but Require Oxidative Metabolism. Cancer Discov. 7,716-735.

Gentles, A. J. (2010). Association of a Leukemic Stem Cell Gene Expression Signature With Clinical Outcomes in Acute Myeloid Leukemia. JAMA 304, 2706.

Hackl, H., Steinleitner, K., Lind, K., Hofer, S., Tosic, N., Pavlovic, S., Suvajdzic, N., Sill, H., and Wieser, R. (2015). A gene expression profile associated with relapse of cytogenetically normal acute myeloid leukemia is enriched for leukemia stem cell genes. Leuk. Lymphoma 56, 1126-1128.

Hallstrom, T. C., Mori, S., and Nevins, J. R. (2008). An E2F1-dependent gene expression program that determines the balance between proliferation and cell death. Cancer Cell 13,11-22.

Heydt, Q., Larrue, C., Saland, E., Bertoli, S., Sarry, J.-E., Besson, A., Manenti, S., Joffre, C., and Mansat-De Mas, V. (2018). Oncogenic FLT3-ITD supports autophagy via ATF4 in acute myeloid leukemia. Oncogene 37,787-797.

Hutchings, C. J., Koglin, M., Olson, W. C., and Marshall, F. H. (2017). Opportunities for therapeutic antibodies directed at G-protein-coupled receptors. Nat. Rev. Drug Discov. 16, 787-810.

wasaki, M., Liedtke, M., Gentles, A. J., and Cleary, M. L. (2015a). CD93 Marks a Non-Quiescent Human Leukemia Stem Cell Population and Is Required for Development of MLL-Rearranged Acute Myeloid Leukemia. Cell Stem Cell 17,412-421.

Khan, I., Halasi, M., Zia, M. F., Gann, P., Gaitonde, S., Mahmud, N., and Gartel, A. L. (2017). Nuclear FOXM1 drives chemoresistance in AML. Leukemia 31, 251-255.

Konopleva, M., Pollyea, D. A., Potluri, J., Chyla, B., Hogdal, L., Busman, T., McKeegan, E., Salem, A. H., Zhu, M., Ricker, J. L., et al. (2016). Efficacy and Biological Correlates of Response in a Phase II Study of Venetoclax Monotherapy in Patients with Acute Myelogenous Leukemia. Cancer Discov. 6, 1106-1117.

Lagadinou, E. D., Sach, A., Callahan, K., Rossi, R. M., Neering, S. J., Minhajuddin, M., Ashton, J. M., Pei, S., Grose, V., O′Dwyer, K. M., et al. (2013). BCL-2 Inhibition Targets Oxidative Phosphorylation and Selectively Eradicates Quiescent Human Leukemia Stem Cells. Cell Stem Cell 12, 329-341.

Leung, J. Y., Ehmann, G. L., Giangrande, P. H., and Nevins, J. R. (2008). A role for Myc in facilitating transcription activation by E2F1. Oncogene 27,4172-4179.

Molina, J. R., Sun, Y., Protopopova, M., Gera, S., Bandi, M., Bristow, C., McAfoos, T., Morlacchi, P., Ackroyd, J., Agip, A.-N.A., et al. (2018). An inhibitor of oxidative phosphorylation exploits cancer vulnerability. Nat. Med. 24, 1036-1046.

Ng, S. W. K., Mitchell, A., Kennedy, J. A., Chen, W.C., McLeod, J., Ibrahimova, N., Arruda, A., Popescu, A., Gupta, V., Schimmer, A. D., et al. (2016). A 17-gene stemness score for rapid determination of risk in acute leukaemia. Nature 540, 433-437.

Nikitenko, L. L., Smith, D. M., Bicknell, R., and Rees, M. C. P. (2003). Transcriptional regulation of the CRLR gene in human microvascular endothelial cells by hypoxia. FASEB J. 17,1499-1501.

O'Donnell, K. A., Wentzel, E. A., Zeller, K. I., Dang, C. V, and Mendell, J. T. (2005). c-Myc-regulated microRNAs modulate E2F1 expression. Nature 435,839-843.

Pei, S., Minhajuddin, M., Adane, B., Khan, N., Stevens, B. M., Mack, S. C., Lai, S., Rich, J. N., Inguva, A., Shannon, K. M., et al. (2018). AMPK/FIS1-Mediated Mitophagy Is Required for Self-Renewal of Human AML Stem Cells. Cell Stem Cell 23, 86-100.e6.

Pellicano, F., Park, L., Hoperoft, L. E. M., Shah, M. M., Jackson, L., Scott, M. T., Clarke, C. J . , Sinclair, A., Abraham, S. A., Hair, A., et al. (2018). hsa-mir183/EGR1-mediated regulation of E2F1 is required for CML stem/progenitor cell survival. Blood 131, 1532-1544.

Rishi, L., Hannon, M., Salome, M., Hasemann, M., Frank, A.-K., Campos, J., Timoney, J., O'Connor, C., Cahill, M. R., Porse, B., et al. (2014). Regulation of Trib2 by an E2F1-C/EBP feedback loop in AML cell proliferation. Blood 123,2389-2400.

Sarry, J., Murphy, K., Perry, R., Sanchez, P. V, Secreto, A., Keefer, C., Swider, C. R., Strzelecki, A., Cavelier, C., Recher, C., et al. (2011). Human acute myelogenous leukemia stem cells are rare and heterogeneous when assayed in NOD/SCID/IL2R γ c-deficient mice. 121.

Schuster, N. M., and Rapoport, A. M. (2017). Calcitonin Gene-Related Peptide-Targeted Therapies for Migraine and Cluster Headache. Clin. Neuropharmacol. 40,169-174.

Suekane, A., Saito, Y., Nakahata, S., Ichikawa, T., Ogoh, H., Tsujikawa, K., and Morishita, K. (2019). CGRP-CRLR/RAMP1 signal is important for stress-induced hematopoiesis. Sci. Rep. 9,429.

Taussig, D. C., Vargaftig, J., Miraki-Moud, F., Griessinger, E., Sharrock, K., Luke, T., Lillington, D., Oakervee, H., Cavenagh, J., Agrawal, S.G., et al. (2010). Leukemia-initiating cells from some acute myeloid leukemia patients with mutated nucleophosmin reside in the CD34(−) fraction. Blood 115,1976-1984.

Van Galen P., Hovestadt V, Wadsworth II M H, Hughes T K (2019). Single-Cell RNA-Seq Reveals A M L Hierarchies Relevant to Disease Progression and Immunity. Cell 1265-1281.e24

Wang, S., Ghosh, R. N., and Chellappan, S. P. (1998). Raf-1 physically interacts with Rb and regulates its function: a link between mitogenic signaling and cell cycle regulation. Mol. Cell. Biol. 18,7487-7498.

Wang, Y., Liu, Y., Malek, S. N., Zheng, P., and Liu, Y. (2011). Targeting HIF1α eliminates cancer stem cells in hematological malignancies. Cell Stem Cell 8,399-411.

(2013). Genomic and Epigenomic Landscapes of Adult De Novo Acute Myeloid Leukemia. N. Engl. J. Med. 368, 2059-2074.

1. A method of depleting leukemic stem cells in a subject suffering from acute myeloid leukemia comprising administering to the subject a therapeutically effective amount of an antibody that specifically binds to CALCRL thereby depleting said leukemic stem cells.

2. A method of depleting leukemic stem cells in a subject suffering from acute myeloid leukemia comprising administering to the subject a therapeutically effective amount of an inhibitor of CALCRL activity or expression thereby depleting said leukemic stem cells.

3. A method of treating chemoresistant acute myeloid leukemia (AML) in a patient in need thereof comprising administering to the patient a therapeutically effective amount an antibody that specifically binds to CALCRL.

4. The method of claim 1, wherein the antibody is a chimeric antibody, a humanized antibody or a human antibody.

5. The method of claim 1, wherein the antibody mediates antibody-dependent cell-mediated cytotoxicity.

6. The method of claim 5 wherein the antibody is an IgG1 antibody.

7. The method of claim 1, wherein the antibody mediates complement dependant cytotoxicity.

8. The method of claim 1, wherein the antibody mediates antibody-dependent phagocytosis.

9. The method of claim 1, wherein the antibody is a multispecific antibody comprising a first antigen binding site directed against CALCRL and at least one second antigen binding site directed against an effector cell.

10. The method of claim 1, wherein the antibody is conjugated to a cytotoxic moiety.

11. A method of treating chemoresistant acute myeloid leukemia (AML) in a patient in need thereof comprising administering to the patient a therapeutically effective amount of an inhibitor of CALCRL activity or expression.

12. The method of claim 11 wherein the inhibitor is a compoun that inhibits the binding of CALCRL to RAMP1 and/or RAMP2 and/or RAMP3.

13. The method of claim 11 wherein the inhibitor inhibits the binding of CALCRL to one of its ligand.

14. The method of claim 11 wherein the inhibitor is an inhibitor of CALCRL, RAMP1, RAMP2, or RAMP3 expression.

15. A method of identifying leukemic stem cells in a sample obtained from a subject suffering from AML comprising identifying in the sample a population of cells that expresses CALCRL and at least one marker of stem cells.

16. The method of claim 15 further comprising identifying the subject as at risk of relapse when the population of cells is identified.

17. The method of claim 12, wherein the inhibitor is an antibody.

18. The method of claim 13, wherein the ligand is adrenomedullin.


SEQUENCE LISTING (1 sequence)

SEQ ID NO: 1 - protein, 461 aa, Homo sapiens
MEKKCTLNFLVLLPFFMILVTAELEESPEDSIQLGVTRNKIMTAQYECYQKIMQDPIQQAEGVYCNRTWDGWLCWNDVAAGTESMQLCPDYFQDFDPSEKVTKICDQDGNWFRHPASNRTWTNYTQCNVNTHEKVKTALNLFYLTIIGHGLSIASLLISLGIFFYFKSLSCQRITLHKNLFFSFVCNSVVTIIHLTAVANNQALVATNPVSCKVSQFIHLYLMGCNYFWMLCEGIYLHTLIVVAVFAEKQHLMWYYFLGWGFPLIPACIHAIARSLYYNDNCWISSDTHLLYIIHGPICAALLVNLFFLLNIVRVLITKLKVTHQAESNLYMKAVRATLILVPLLGIEFVLIPWRPEGKIAEEVYDYIMHILMHFQGLLVSTIFCFFNGEVQAILRRNWNQYKIQFGNSFSNSEALRSASYTVSTISDGPGYSHDCPSEHLNGKSIHDIENVLLKPENLYN